(19) United States
(12) Patent Application Publication
(10) Pub. No.: US 2021/0235968 A1
(43) Pub. Date: Aug. 5, 2021

(54) MEDICAL SYSTEM, INFORMATION PROCESSING APPARATUS, AND INFORMATION PROCESSING METHOD

(71) Applicant: SONY CORPORATION, TOKYO (JP)

(72) Inventors: KENTARO FUKAZAWA, TOKYO (JP); DAISUKE KIKUCHI, TOKYO (JP)

(21) Appl. No.: 17/250,669

(22) PCT Filed: Aug. 7, 2019
(86) PCT No.: PCT/JP2019/031245

§ 371 Date: Feb. 17, 2021

(30) Foreign Application Priority Data
Aug. 28, 2018 (JP) 2018-159676

(51) Int. Cl. A61B 1/00; A61B 1/06; G02B 21/00; G02B 27/48

(52) U.S. Cl. CPC A61B 1/00009; G02B 27/48; G02B 21/0012; A61B 1/0661

(57) ABSTRACT

In a medical system (1), speckle contrast calculation means (1313, 1314) calculates a first speckle contrast value for each pixel based on a first speckle image at a first exposure time and/or a second speckle contrast value for each pixel based on a second speckle image at a second exposure time shorter than the first exposure time. In addition, speckle image generation means (1315) generates a speckle contrast image on the basis of the first speckle contrast value and/or the second speckle contrast value depending on a detection result of motion of an image capturing target by motion detection means (1312).

### FIELD

The present disclosure relates to a medical system, an information processing apparatus, and an information processing method.

### BACKGROUND

In the field of medical systems, in one example, speckle imaging technology that enables constant observation of blood flow or lymph flow has been developed. In this technology, a speckle is a phenomenon in which a speckle pattern occurs, in one example, due to reflections or interferences of irradiated coherent light from minute irregularities on a target object's surface. The use of such a speckle phenomenon allows for, in one example, discrimination between a part where blood flow (blood flow part) and a part where blood does not flow (non-blood flow part) in the living body as a target object.

The specific details are as follows. In the case of increasing the exposure time to some extent, a speckle contrast value decreases due to the movement of red blood cells or other blood products that reflect coherent light in the blood flow part, whereas the speckle contrast value increases in the non-blood flow part because all elements are in the non-flowing state. Thus, a speckle contrast image generated using a speckle contrast value of each pixel allows for discrimination between the blood flow and non-blood flow parts.

### CITATION LIST

### Patent Literature

Patent Literature 1: JP 2016-193066 A

### SUMMARY

### Technical Problem

However, in using the speckle imaging technology, sometimes, the living body that is a target object moves due to body movement, pulsation, or the like, or an image capturing apparatus shakes for some reason. In this case, the entirety or a part of an image capturing target in a captured image will move, causing the speckle contrast value of the non-blood flow part to greatly decrease. Thus, the discrimination accuracy between the blood flow and non-blood flow parts sometimes deteriorates. In addition, shortening the exposure time makes it possible to reduce the decrease in the speckle contrast value of the non-blood flow part in the case where the image capturing target moves. While in this case, a decrease in the amount of light will cause the signal-noise ratio (S/N) to deteriorate, leading to reduced accuracy of discrimination between the blood flow and non-blood flow parts.

Thus, the present disclosure provides a medical system, information processing apparatus, and information processing method, capable of generating a satisfactory speckle contrast image even in the case the image capturing target moves in the captured image in using the speckle imaging technology.

### Solution to Problem

To solve the problem described above, a medical system according to one aspect of the present disclosure includes light irradiation means for irradiating an image capturing target with coherent light, image capturing means for capturing a speckle image obtained from scattered light caused by the image capturing target irradiated with the coherent light, acquisition means for acquiring a first speckle image at a first exposure time and a second speckle image at a second exposure time shorter than the first exposure time, speckle contrast calculation means for calculating a first speckle contrast value for each pixel based on the first speckle image and/or a second speckle contrast value for each pixel based on the second speckle image, motion detection means for detecting motion of the image capturing target, and speckle image generation means for generating a speckle contrast image on a basis of the first speckle contrast value and/or the second speckle contrast value depending on a detection result of the motion of the image capturing target by the motion detection means.

### BRIEF DESCRIPTION OF DRAWINGS

FIG. 1 is a diagram illustrating an exemplary configuration of a medical system according to the first embodiment of the present disclosure.

FIG. 2 is a diagram illustrating an exemplary configuration of an information processing apparatus according to the first embodiment of the present disclosure.

FIG. 3 is a diagram illustrated to describe a technique using spatial-division two-step exposure according to the first embodiment of the present disclosure.

FIG. 4 is a diagram illustrated to describe a technique using temporal-division two-step exposure according to the first embodiment of the present disclosure.

FIG. 5 is a diagram illustrated to describe a technique using light-ray split two-step exposure according to the first embodiment of the present disclosure.

FIG. 6 is a diagram illustrated to describe a technique using high frame rate imaging according to the first embodiment of the present disclosure.

FIG. 7 is a diagram illustrated to describe the relationship between a mixing ratio of first SC and second SC in a second method of using SC according to the first embodiment of the present disclosure.

FIG. 8 is a flowchart illustrating the first SC image generation processing performed by the information processing apparatus according to the first embodiment of the present disclosure.

FIG. 9 is a flowchart illustrating the second SC image generation processing performed by the information processing apparatus according to the first embodiment of the present disclosure.

FIG. 10 is a diagram illustrating an exemplary configuration of an information processing apparatus according to a second embodiment of the present disclosure.

FIG. 11 is a graph illustrating SC upon long-time exposure and SC upon short-time exposure of each of a fluid part and a non-fluid part according to the second embodiment of the present disclosure.

FIG. 12 is a flowchart illustrating the third SC image generation processing performed by the information processing apparatus according to the second embodiment of the present disclosure.

FIG. 13 is a flowchart illustrating the fourth SC image generation processing performed by the information processing apparatus according to the second embodiment of the present disclosure.

FIG. 14 is a diagram illustrating an exemplary configuration of an information processing apparatus according to a third embodiment of the present disclosure.

FIG. 15 is a diagram illustrated to describe a technique using spatial-division two-step exposure and a technique using temporal-division two-step exposure according to the third embodiment of the present disclosure.

FIG. 16 is a view illustrating an example of a schematic configuration of an endoscopic surgery system according to application example 1 of the present disclosure.

FIG. 17 is a block diagram illustrating an example of a functional configuration of a camera head and a CCU illustrated in FIG. 16.

FIG. 18 is a view illustrating an example of a schematic configuration of a microscopic surgery system according to application example 2 of the present disclosure.

FIG. 19 is a view illustrating a state of surgery in which the microscopic surgery system illustrated in FIG. 18 is used.

FIG. 20 is a graph illustrating SC upon long-time exposure and SC upon short-time exposure of each of a fluid part and a non-fluid part according to the first embodiment of the present disclosure.

FIG. 21 is a diagram illustrating an example of an SC image of a pseudo blood vessel in the first embodiment of the present disclosure.

### DESCRIPTION OF EMBODIMENTS

The description is now given of embodiments of the present disclosure in detail with reference to the drawings. Moreover, in embodiments described below, the same components are denoted by the same reference numerals, and so a description thereof is omitted as appropriate.

In neurosurgical procedure and cardiac surgical procedure, fluorescence observation using indocyanine green (ICG) is generally performed for blood flow observation at the surgery. This ICG fluorescence observation is a technique of observing the circulation of blood or lymphatic vessels in a minimally invasive manner by utilizing the characteristics that ICG binds to plasma protein in vivo and emits fluorescence by near-infrared excitation light.

The ICG fluorescence observation technique necessitates the administration of an appropriate amount of ICG to the living body in advance in accordance with the observation timing. In the case of repeated observation, the in vitro release of ICG is necessary to wait. Thus, this waiting for observation makes rapid medical treatment difficult, and there is a possibility of causing a delay in surgery. Furthermore, the ICG observation makes the presence or absence of blood or lymph vessels recognizable but fails to observe the presence or absence or the speed of blood or lymph flow.

Thus, in consideration of the above-described situation, a speckle imaging technology capable of making the administration of drugs unnecessary and enabling constant observation of blood or lymph flows is developed. Specific application examples include aneurysm occlusion evaluation in cerebral aneurysm clipping surgery. In cerebral aneurysm clipping surgery using ICG observation, ICG is injected after clipping to determine the presence or absence of aneurysm occlusion. However, if ICG is injected when the occlusion is not enough to diagnose, the ICG will flow into the aneurysm. Thus, the occlusion evaluation is inaccurate in some cases due to the remaining ICG when clipping is performed again. On the other hand, in cerebral aneurysm clipping surgery using blood flow observation based on speckle, the presence or absence of aneurysm occlusion fails to be repeatedly determined with high accuracy without using a drug.

Hereinafter, explanation will be given regarding a medical system, information processing apparatus, and information processing method, capable of generating a satisfactory speckle contrast image even in the case the image capturing target moves in the captured image in using the speckle imaging technology.

### First Embodiment

### [Medical System According to First Embodiment]

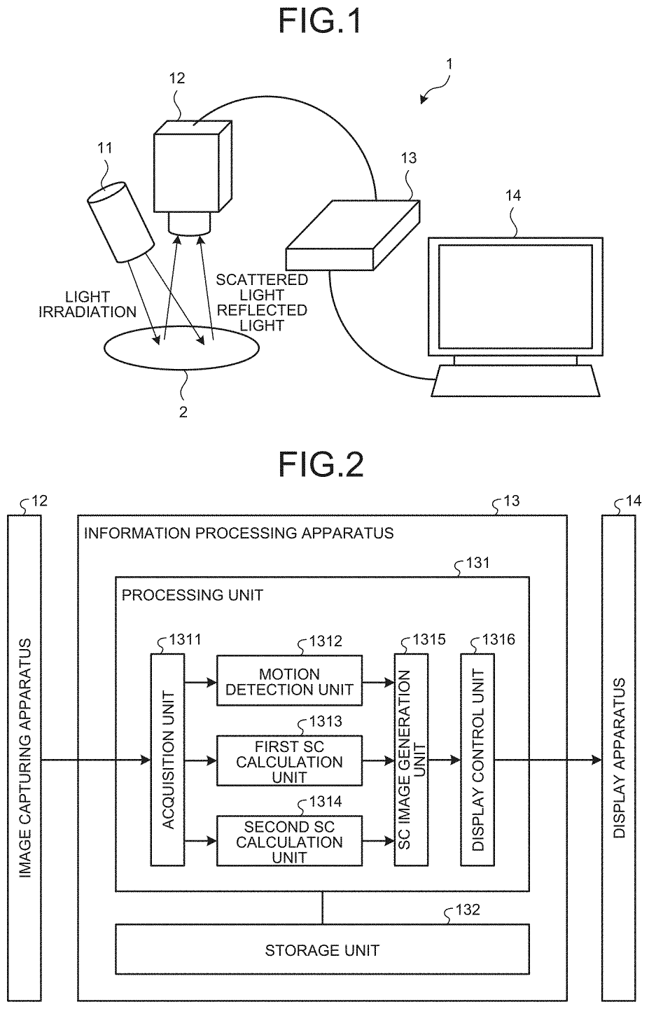

FIG. 1 is a diagram illustrating an exemplary configuration of a medical system 1 according to the first embodiment of the present disclosure. The medical system 1 according to the first embodiment roughly includes at least a light source 11 (light irradiation means), an image capturing apparatus 12, and an information processing apparatus 13. In addition, a display apparatus 14 or the like can be further provided if necessary. Each component is now described in detail.

(1) Light Source

The light source 11 includes a first light source that irradiates an image capturing target with coherent light used to capture a speckle image. The coherent light is a beam or ray indicating that the phase relation of light waves at any two points in luminous flux is invariant and constant over time, and it shows perfect coherence even in splitting the luminous flux by any method and then superposing it again with a substantial optical path difference. The wavelength of the coherent light output from the first light source according to the present disclosure is preferably, in one example, 830 nm. This is because, if the wavelength is 830 nm, the ICG observation and an optical system can be used together. In other words, it is common to use near-infrared light with a wavelength of 830 nm in the case where performing the ICG observation. Thus, this is because near-infrared light of the same wavelength used for speckle observation enables the speckle observation to be performed without modifying the optical system of a microscope capable of performing the ICG observation.

Note that the wavelength of the coherent light emitted by the first light source is not limited to the example described above, and examples of wavelength can include, in one instance, 550 to 700 nm or other wavelengths. The description below is given, as an example, a case where the near-infrared light having a wavelength of 830 nm is employed as coherent light.

Further, the type of the first light source that emits coherent light is not limited to a particular one as long as the effect of the present technology is not impaired. Examples of the first light source that emits laser light include an argon (Ar) ion laser, a helium-neon (He—Ne) laser, a dye laser, a krypton (Cr) laser, a semiconductor laser, a solid-state laser in which a semiconductor laser and wavelength conversion optics are combined, or the like, which of each can be used alone or in combination with each other.

Further, the light source 11 may include a second light source that irradiates an image capturing target 2 with visible light used to capture a visible-light image (e.g., white light of incoherent light). In this case, an image capturing target 2 is irradiated simultaneously with coherent light and visible light. In other words, the second light source emits light at the same time as the first light source. In this description, incoherent light refers to light that rarely exhibits coherence, such as an object beam (object waves). The type of the second light source is not limited to a particular one as long as the effect of the present technology is not impaired. An example thereof can include a light-emitting diode or the like. In addition, other examples of the light source include a xenon lamp, a metal halide lamp, a high-pressure mercury lamp, or the like.

(2) Image Capturing Target

The image capturing target 2 can be various, but in one example, one containing a fluid is preferable. Due to the speckle characteristics, the speckle unlikely occurs from fluid. Thus, forming an image of the image capturing target 2 having fluidal substances using the medical system 1 according to the present disclosure allows for obtaining the boundary between a fluid part and a non-fluid part, flow rate of the fluid part, or the like.

More specifically, in one example, the image capturing target 2 can be a living body whose fluid is blood. In one example, the use of the medical system 1 according to the present disclosure in microscopic surgery, endoscopic surgery, or the like makes it possible to perform surgery while checking the blood vessels' position. Thus, it is possible to perform safer and more accurate surgery, leading to a contribution to the further development of medical technology.

(3) Image Capturing Apparatus

The image capturing apparatus 12 includes a speckle image capturing unit (image capturing means) that captures a speckle image obtained from scattered light (reflected light can be included) caused by the image capturing target 2 irradiated with coherent light. The speckle image capturing unit is, in one example, an infrared (IR) imager for speckle observation.

Further, the image capturing apparatus 12 can include a visible-light image capturing unit. The visible-light image capturing unit is, in one example, an RGB (red/green/blue) imager for visible-light observation. In this case, the image capturing apparatus 12 includes, in one example, a dichroic mirror as the main configuration in addition to the speckle image capturing unit and the visible-light image capturing unit. In addition, the light source 11 emits near-infrared light and visible light. The dichroic mirror separates the received light into near-infrared light (scattered light or reflected light) and visible light (scattered light or reflected light). The visible-light image capturing unit captures a visible-light image obtained from visible light separated by the dichroic mirror. With the image capturing apparatus 12 having such a configuration, it is possible to simultaneously perform speckle observation using near-infrared light and visible-light observation using visible light. Moreover, the speckle image and the visible-light image can be captured by the respective individual image capturing apparatuses.

(4) Information Processing Apparatus

The description is now given of the information processing apparatus 13 with reference to FIG. 2. FIG. 2 is a diagram illustrating an exemplary configuration of the information processing apparatus 13 according to the first embodiment of the present disclosure. The information processing apparatus 13 is an image processing apparatus and mainly includes a processing unit 131 and a storage unit 132. Moreover, “SC” herein refers to speckle contrast (speckle contrast value).

The processing unit 131 is configured with, in one example, a central processing unit (CPU). The processing unit 131 includes an acquisition unit 1311 (acquisition means), a motion detection unit 1312 (motion detection means), a first SC calculation unit 1313 (speckle contrast calculation means), a second SC calculation unit 1314 (speckle contrast calculation means), an SC image generation unit 1315 (speckle image generation means), and a display control unit 1316.

The acquisition unit 1311 acquires a first speckle image at a first exposure time, and further acquires a second speckle image at a second exposure time shorter than the first exposure time (details thereof later).

The motion detection unit 1312 detects the motion of the image capturing target 2. The motion detection unit 1312 calculates a motion vector on the basis of a difference between the current frame and the immediately preceding frame on the basis of, in one example, the speckle image or the visible-light image. If the absolute value of the motion vector is equal to or higher than a predetermined difference threshold, the motion detection unit 1312 determines that the image capturing target 2 moves. This motion detection can be performed for each pixel, each block, or the entire screen. In addition, it is also possible to detect the amount of movement (0 pixels, 1 pixel, 2 pixels, . . . ) rather than determining whether or not there is movement. In addition, the motion detection unit 1312 can detect the motion or the amount of movement of the image capturing target 2, in one example, by using the property that the speckle's shape extends in the direction of movement upon motion of the image capturing target 2, in addition to the detection using the motion vector.

The first SC calculation unit 1313 calculates a first speckle contrast value for each pixel on the basis of a first speckle image. In this regard, in one example, a speckle contrast value of i-th pixel can be expressed by Formula (1) as follows:

Speckle contrast value of i-th pixel=(standard deviation of intensity between i-th pixel and adjacent pixels)/(mean of intensity of i-th pixel and adjacent pixels) Formula (1)

The second SC calculation unit 1314 calculates a second speckle contrast value for each pixel on the basis of the second speckle image. A method of calculation is similar to that of the first SC calculation unit 1313.

The SC image generation unit 1315 generates a speckle contrast image on the basis of the first speckle contrast value and/or the second speckle contrast value depending on a detection result of the motion of the image capturing target 2 by the motion detection unit 1312 (details thereof later). An example of the SC image is now described with reference to FIG. 21. FIG. 21 is a diagram illustrating an exemplary SC image of a pseudo blood vessel according to the first embodiment of the present disclosure. As illustrated in the SC image example of FIG. 21, many speckles are observed in the non-blood flow part, but speckles are hardly observed in the blood flow part.

Referring back to FIG. 2, the SC image generation unit 1315 discriminates between a fluid part (e.g., a blood flow part) and a non-fluid part (e.g., a non-blood flow part) on the basis of the SC image. More specifically, the SC image generation unit 1315 discriminates between the blood flow part and the non-blood flow part by determining whether or not the speckle contrast value is equal to or higher than a predetermined SC threshold on the basis of the SC image.

The display control unit 1316 controls the display apparatus 14 to display the SC image so that the blood flow part and the non-blood flow part can be discriminated from each other, on the basis of the SC image generated by the SC image generation unit 1315.

The storage unit 132 stores various types of information such as speckle images acquired by the acquisition unit 1311, visible-light images, and calculation results by each component of the processing unit 131. Moreover, an external storage device of the medical system 1 can be used instead of the storage unit 132.

(5) Display Apparatus

The display apparatus 14 displays various types of information such as the speckle image acquired by the acquisition unit 1311, the visible-light image, and calculation results by each unit of the processing unit 131 under the control of the display control unit 1316. Moreover, an external display apparatus of the medical system 1 can be used instead of the display apparatus 14.

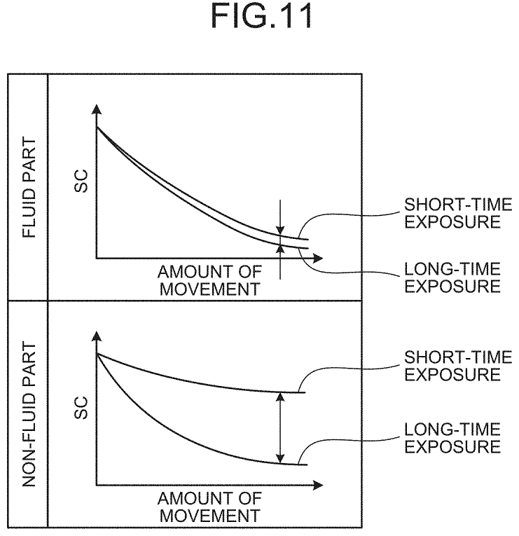

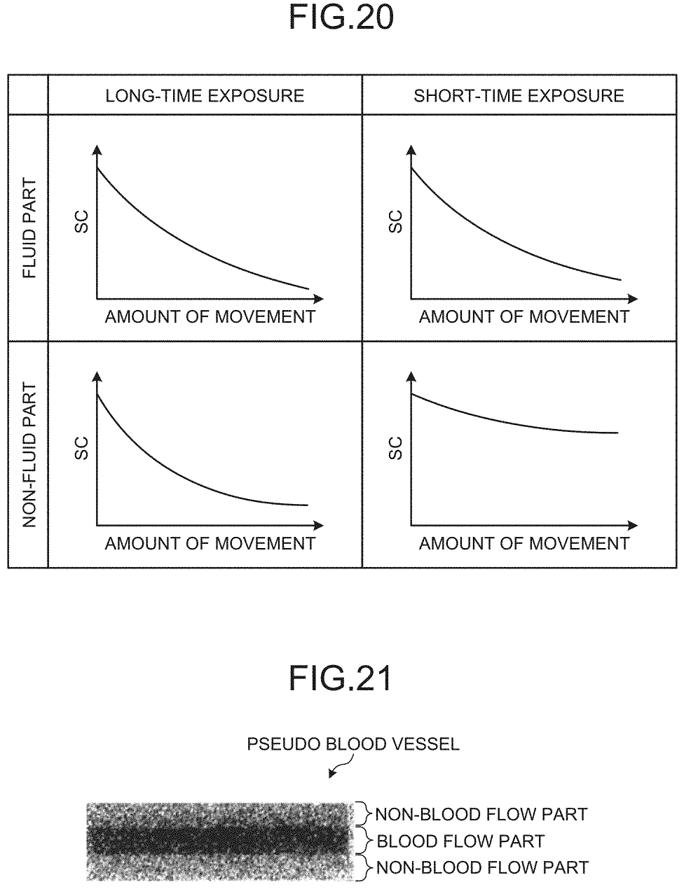

The description is now given of SC upon long-time exposure and SC upon short-time exposure of each of the fluid part and the non-fluid part with reference to FIG. 20. FIG. 20 is a graph illustrating SC upon long-time exposure and SC upon short-time exposure of each of the fluid part and the non-fluid part according to the first embodiment of the present disclosure. Moreover, blood is herein assumed as a fluid (the same applies to FIG. 11). Then, red blood cells or other blood products precipitate in the blood in a non-flowing state, so the SC of the fluid part and the SC of the non-fluid part are similar in the non-flowing state.

As can be seen from FIG. 20, first, for the fluid part, the SC is high when the amount of movement is small in both the long-time exposure and the short-time exposure. Besides, with the increase in the amount of movement, both the SC of long-time exposure and the SC of short-time exposure decrease. Then, in the case where the amounts of movement are large and the same, the SC of short-time exposure is higher than the SC of long-time exposure, but the difference between the two is small.

On the other hand, for the non-fluid part, the SC is high when the amount of movement is small in both the long-time exposure and the short-time exposure. Besides, with the increase in the amount of movement, both the SC of long-time exposure and the SC of short-time exposure decrease. Then, in the case where the amounts of movement are large and the same, the SC of short-time exposure is higher than the SC of long-time exposure, and the difference between the two is big.

In other words, for the long-time exposure, the amount of light is large, and so the S/N is satisfactory, but if the image capturing target 2 moves, the SC of the non-fluid part is greatly reduced, causing the difference between the SC of the fluid part and the SC of the non-fluid part to be smaller. On the other hand, for the short-time exposure, even if the image capturing target 2 moves, the amount of decrease in SC of the non-fluid part can be suppressed to a small extent, and so the difference between the SC in the fluid part and the SC in the non-fluid part can be increased. However, the amount of light is small, and so the S/N is not satisfactory. Then, the description of the present disclosure is herein given of a technique of combining the advantages of long-time exposure and short-time exposure.

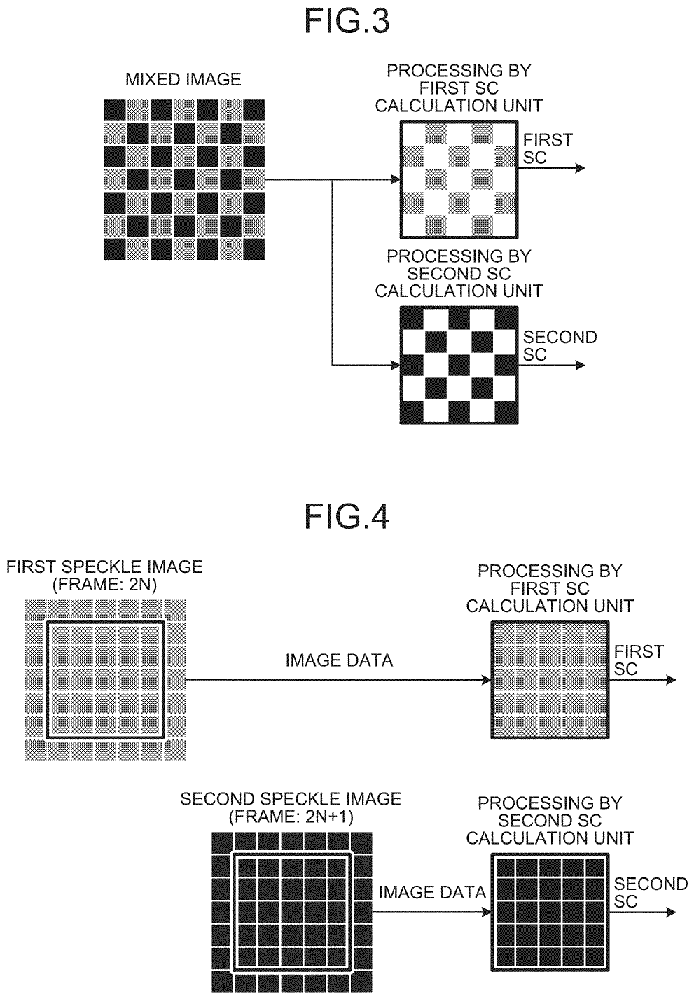

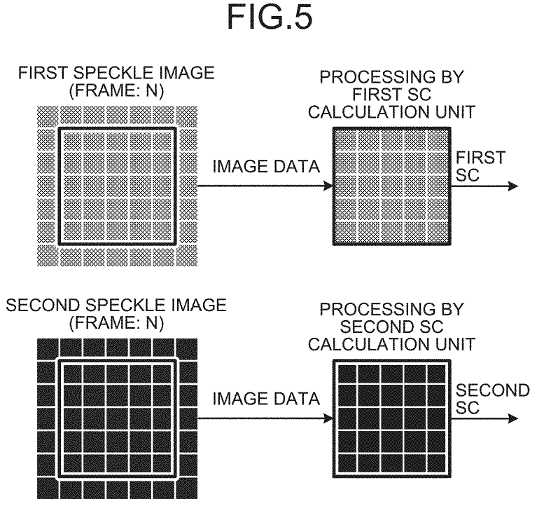

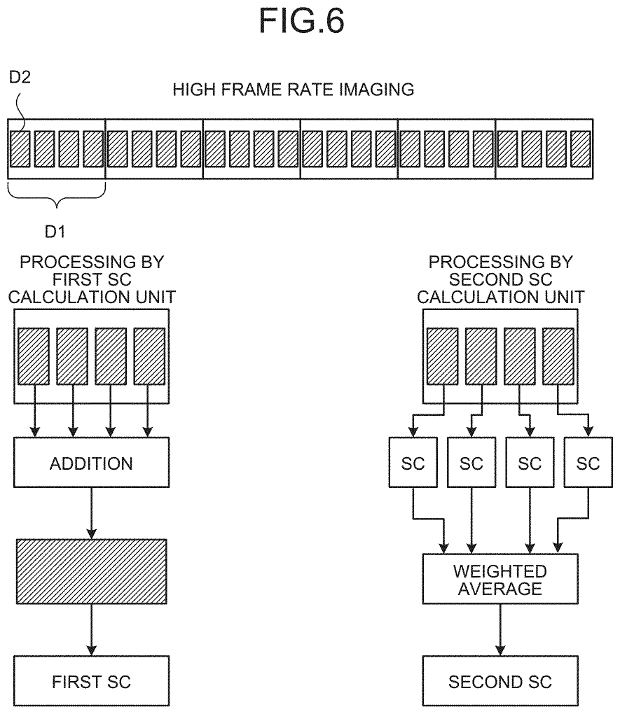

The description is now given of a specific technique of calculating two types of SCs on the basis of two speckle images (a first speckle image at a first exposure time and a second speckle image at a second exposure time) at two types of exposure time (the first exposure time and the second exposure time shorter than the first exposure time). The description is now given of a technique using spatial-division two-step exposure (an example of spatial-division multi-step exposure) with reference to FIG. 3. The description is also given of a technique using temporal-division two-step exposure (an example of temporal-division multi-step exposure) with reference to FIG. 4. Besides, the description is given of a technique using light-ray split two-step exposure (an example of ray split multi-step exposure) with reference to FIG. 5. In addition, the description is given of a technique using high frame rate imaging with reference to FIG. 6.

FIG. 3 is a diagram illustrated to describe a technique using spatial-division two-step exposure according to the first embodiment of the present disclosure. In this technique, the image capturing apparatus 12 captures, in one example, a mixed image illustrated in FIG. 3. In this mixed image, a pixel of the first speckle image (hereinafter refers to “first S pixel”) and a pixel of the second speckle image (hereinafter refers to “second S pixel”) are alternately included in both the vertical and horizontal directions in one frame. In the mixed image of FIG. 3, the lighter-colored pixel is the first S pixel, and the darker-colored pixel is the second S pixel. The acquisition unit 1311 acquires such mixed pixels from the image capturing apparatus 12.

Further, the first SC calculation unit 1313 calculates the first speckle contrast value (hereinafter refers to “first SC”) for each pixel on the basis of the first S pixel in the mixed image. In addition, the second SC calculation unit 1314 calculates the second speckle contrast value (hereinafter refers to “second SC”) for each pixel on the basis of the second S pixel in the mixed image. In this way, it is possible to calculate two types of SCs (first and second SCs).

Next, FIG. 4 is a diagram illustrated to describe a technique using temporal-division two-step exposure according to the first embodiment of the present disclosure. In this technique, the acquisition unit 1311 alternately acquires the first speckle image (frame: 2N) and the second speckle image (frame: 2N+1) in a time-series order. For this purpose, in one example, the single image capturing apparatus 12 switches between the image capturing of the first speckle image and the image capturing of the second speckle image.

Further, the first SC calculation unit 1313 calculates the first SC for each pixel on the basis of the first speckle image. In addition, the second SC calculation unit 1314 calculates the second SC for each pixel on the basis of the second speckle image. In this way, it is possible to calculate two types of SCs (first and second SCs).

Moreover, this technique necessitates two frames of speckle images to calculate a set of the first SC and the second SC. Thus, the frame rate of the SC image created by using the first SC and the second SC can be any of those described below. In one example, the frame rate of the SC image is one-half of an image-capturing frame rate of the speckle image. Alternatively, it can be the same as the image-capturing frame rate of the speckle image by outputting the same SC image for two consecutive frames. Furthermore, it can be the same as the image-capturing frame rate of the speckle image by interpolating the frame rate of the SC image from the preceding and following SC images to generate the SC image between them.

Next, FIG. 5 is a diagram illustrated to describe a technique using light-ray split two-step exposure according to the first embodiment of the present disclosure. In this technique, the incident light is optically branched, and two image capturing apparatuses 12 having different exposure time periods (e.g., a first image capturing apparatus and a second image capturing apparatus having a shorter exposure time than the first image capturing apparatus) capture the first speckle image and the second speckle image, respectively. Then, the acquisition unit 1311 acquires the first speckle image (frame: N) and the second speckle image (frame: N) from these two image capturing apparatuses.

Further, the first SC calculation unit 1313 calculates the first SC for each pixel on the basis of the first speckle image. In addition, the second SC calculation unit 1314 calculates the second SC for each pixel on the basis of the second speckle image. In this way, it is possible to calculate two types of SCs (first and second SCs).

Next, FIG. 6 is a diagram illustrated to describe a technique using high frame rate imaging according to the first embodiment of the present disclosure. In this technique, in one example, a speckle image is captured at four times the normal speed. In FIG. 6, a time duration D1 is a unit time for normal imaging, and a time duration D2 is a unit time for high frame rate imaging. The acquisition unit 1311 acquires a high frame rate speckle image from the image capturing apparatus 12.

Further, the first SC calculation unit 1313 calculates the first SC by using a plurality of frames (here, four frames) of a high frame rate speckle image. In one example, the first SC calculation unit 1313 adds four frames of a high frame rate speckle image to make the state equivalent in terms of exposure time compared with a normal frame rate and then calculates the first SC.

Further, the second SC calculation unit 1314 calculates the second SC using one frame of a high frame rate speckle image. In one example, the second SC calculation unit 1314 calculates the second SC by calculating each SC for four frames of a high frame rate speckle image and calculating the weighted average on each SC. In this way, it is possible to calculate two types of SCs (first and second SCs).

The description is now given of a method of using the first SC and a method of using the second SC in the SC image generation unit 1315. In one example, in the first method of using SC, the SC image generation unit 1315 generates an SC image by using the first SC in the case where the motion of the image capturing target 2 is not detected by the motion detection unit 1312. The SC image generation unit 1315 generates an SC image by using the second SC in the case where the motion of the image capturing target 2 is detected by the motion detection unit 1312.

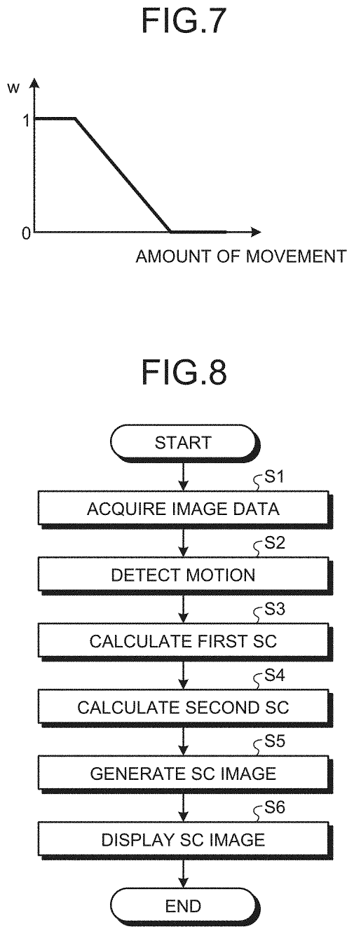

Then, in the second method of using SC, the SC image generation unit 1315 generates an SC image by using an SC obtained by weighting and summing and synthesizing the first SC and the second SC (hereinafter also refers to “synthetic Sc”). This generation is based on the amount of movement of the image capturing target 2 detected by the motion detection unit 1312. This is described with reference to FIG. 7. FIG. 7 is a diagram illustrated to describe the relationship between mixing ratios of the first SC and the second SC in the second method of using SC according to the first embodiment of the present disclosure. A coefficient w (mixing ratio) depending on the amount of movement of the image capturing target 2 is set as illustrated in FIG. 7. In the graph of FIG. 7, the vertical axis represents the value of w, and the horizontal axis represents the amount of movement of the image capturing target 2. Then, the SC image generation unit 1315 calculates a synthetic SC by Formula (2) as follows:

Synthetic SC=w×1st SC+(1−w)×2nd SC Formula (2)

As described above, if the amount of movement of the image capturing target 2 is small, the ratio of the first SC increases, and if the amount of movement of the image capturing target 2 is large, the ratio of the second SC increases, making the synthetic SC adequate.

[Information Processing Apparatus According to First Embodiment]

The description is now given of the first SC image generation processing performed by the information processing apparatus 13 with reference to FIG. 8. FIG. 8 is a flowchart illustrating the first SC image generation processing performed by the information processing apparatus 13 according to the first embodiment of the present disclosure.

In step S1, the acquisition unit 1311 initially acquires image data. In one example, for the technique using spatial-division two-step exposure, the mixed image is acquired (FIG. 3). In addition, for the technique of using temporal-division two-step exposure, the first speckle image and the second speckle image are acquired (FIG. 4). Besides, for the technique of using light-ray split two-step exposure, the first speckle image and the second speckle image are acquired (FIG. 5). In addition, for the technique of using high frame rate imaging, a high frame rate speckle image is acquired (FIG. 6).

Subsequently, in step S2, the motion detection unit 1312 detects the motion of the image capturing target 2.

Subsequently, in step S3, the first SC calculation unit 1313 calculates the first SC for each pixel on the basis of the first speckle image.

Subsequently, in step S4, the first SC calculation unit 1313 calculates the second SC for each pixel on the basis of the second speckle image. Moreover, the processing details in each of the four techniques in steps S3 and S4 are already described with reference to FIGS. 3 to 6.

Subsequently, in step S5, the SC image generation unit 1315 generates a speckle contrast image on the basis of the first SC and the second SC depending on a result obtained by detecting the motion of the image capturing target 2 in step S2. Specifically, according to the first method of using SC and the second method of using SC as described above, in one example, the SC image generation unit 1315 generates the SC image using the first SC and the second SC on the basis of the presence or absence of motion and the amount of movement of the image capturing target 2.

Further, in step S5, the SC image generation unit 1315 discriminates between the blood flow part and the non-blood flow part on the basis of, in one example, the SC image.

Subsequently, in step S6, the display control unit 1316 controls the display apparatus 14 to display the SC image so that the blood flow part and the non-blood flow part can be discriminated on the basis of the SC image generated in step S5. After step S6, the processing ends.

As described above, according to the first SC image generation processing by the information processing apparatus 13 of the first embodiment, it is possible to generate a satisfactory speckle contrast image even in the case where the image capturing target 2 moves in the captured image. Specifically, in one example, in the case where the motion of the image capturing target 2 is not detected by the above-mentioned first method of using the SC, the first SC is used to generate the SC image. In the case where the motion detection unit 1312 detects the motion of the image capturing target 2, the second SC is used to generate the SC image. In addition, in one example, according to the above-described second method of using SC, the SC image is generated using the SC obtained by weighting and summing and synthesizing the first SC and the second SC on the basis of the amount of movement of the image capturing target 2. This makes it possible to reduce the decrease in SC in the non-blood flow part even in the case where the image capturing target 2 moves. In addition, in the case where the image capturing target 2 does not move, it is possible to avoid S/N deterioration due to the shortening of the exposure time.

Moreover, the motion detection, motion speed calculation, and SC calculation associated therewith of the image capturing target 2 can be performed on the captured entire screen, or can be performed for each region, in one example, after previously analyzing color information or morphological information of the visible-light image and discriminating between the blood vessel and non-blood vessel portions.

Further, the motion detection and the motion speed calculation of the image capturing target 2 can be easily achieved, in one example, by calculating the motion vector of the feature points on the basis of a plurality of visible-light images in a time series.

Further, the motion detection and the motion speed calculation of the image capturing target 2 can be easily achieved, in one example, by recognizing fluctuation in the shapes of speckles on the basis of the speckle image.

Note that, in the related art, there is a technique of reducing the influence of fluctuation in speckle patterns due to the motion of an image capturing target by shortening the exposure time. However, this technique necessitates complicated control mechanisms for synchronous control of an illumination unit and an image capturing unit. It also necessities a high-power laser light source for observation under low exposure conditions, leading to difficulties in achieving it. On the other hand, the present disclosure makes such a complicated control mechanism and a high-power laser light source unnecessary.

Further, according to the technique of using spatial-division two-step exposure (FIG. 3), it is possible to calculate two types of SCs (the first and second SCs) from one mixed image having the first S pixel and the second S pixel.

Then, according to the technique of using temporal-division two-step exposure (FIG. 4), switching between the image capturing of the first speckle image and the image capturing of the second speckle image by a single image capturing apparatus 12 makes it possible to calculate two types of SCs (the first and second SCs).

Further, according to the technique of using light-ray split two-step exposure (FIG. 5), it is possible to eliminate the need to reduce the calculation frequency of two types of SCs (the first and second SCs) by allowing the first speckle image and the second speckle image to be acquired simultaneously.

Then, according to the technique of using high frame rate imaging (FIG. 6), it is possible to calculate two types of SCs (the first and second SCs) on the basis of one high frame rate speckle image.

Moreover, in the case of performing speckle observation in the medical field, there is an upper limit to the amount of light emitted from the light source even if it is strengthened. This is because if the amount of light is too strong, the affected part can be damaged, or the surgeon's eyes can be damaged. According to the medical system 1 of the first embodiment, the need to increase the amount of light is eliminated. In other words, the amount of light emitted by the light source 11 can be the same when the image capturing apparatus 12 captures the first speckle image and captures the second speckle image.

However, the amount of light upon capturing the second speckle image having the shorter exposure time can be larger than the amount of light upon capturing the first speckle image to the extent that it does not affect. By doing so, it is possible to reduce the deterioration of S/N due to a short exposure time upon capturing the second speckle image.

Further, steps S2, S3, and S4 in FIG. 8 are not limited to the order described above, and they can be optionally reordered.

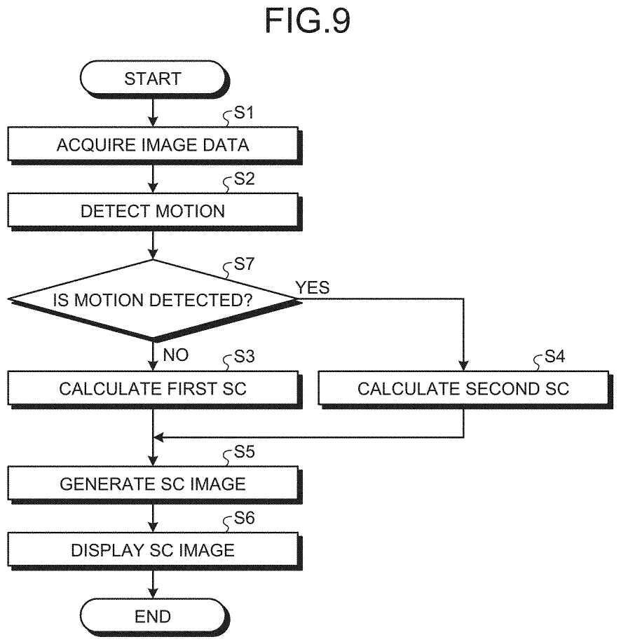

The description is now given of the second SC image generation processing performed by the information processing apparatus 13 with reference to FIG. 9. FIG. 9 is a flowchart illustrating the second SC image generation processing performed by the information processing apparatus 13 according to the first embodiment of the present disclosure. The description of the matters similar to FIG. 8 will be omitted as appropriate.

In step S1, the acquisition unit 1311 initially acquires image data.

Subsequently, in step S2, the motion detection unit 1312 performs an operation for detecting the motion of the image capturing target 2.

Subsequently, in step S7, the motion detection unit 1312 determines whether or not the image capturing target 2 is moved. If the result is Yes, the processing proceeds to step S4. If the result is No, the processing proceeds to step S3.

Subsequently, in step S3, the first SC calculation unit 1313 calculates the first SC for each pixel on the basis of the first speckle image. Then, in step S5, the SC image generation unit 1315 generates an SC image on the basis of the first SC calculated in step S3.

Subsequently, in step S4, the first SC calculation unit 1313 calculates the second SC for each pixel on the basis of the second speckle image. Then, in step S5, the SC image generation unit 1315 generates an SC image on the basis of the second SC calculated in step S4.

Further, in step S5, the SC image generation unit 1315 discriminates between the blood flow part and the non-blood flow part on the basis of, in one example, the SC image.

Subsequently, in step S6, the display control unit 1316 controls the display apparatus 14 to display the SC image so that the blood flow part and the non-blood flow part can be discriminated on the basis of the SC image generated in step S5. After step S6, the processing ends.

As described above, according to the second SC image generation processing by the information processing apparatus 13 of the first embodiment, it is possible to generate the SC image by calculating only one of the first and second SCs depending on the presence or absence of motion of the image capturing target 2, allowing simplifying the processing.

### Second Embodiment

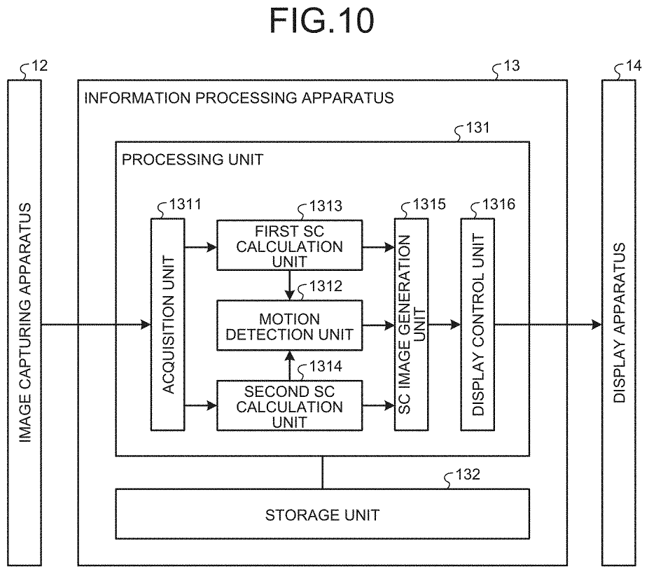

A second embodiment is now described. The same matters as in the first embodiment will be omitted as appropriate. FIG. 10 is a diagram illustrating an exemplary configuration of an information processing apparatus 13 according to the second embodiment of the present disclosure. In the information processing apparatus 13 according to the second embodiment, the motion detection unit 1312 detects the motion of the image capturing target 2 on the basis of a value obtained by subtracting the first SC from the second SC.

FIG. 11 is herein a graph illustrating the SC upon long-time exposure and the SC upon short-time exposure for each of the fluid part and the non-fluid part according to the second embodiment of the present disclosure. As can be seen from FIG. 11, for the fluid part, in the case where the amount of movement of the image capturing target 2 is large, the value obtained by subtracting the first SC (long-time exposure) from the second SC (short-time exposure) is small. On the other hand, for the non-fluid part, in the case where the amount of movement of the image capturing target 2 is large, the value obtained by subtracting the first SC (long-time exposure) from the second SC (short-time exposure) (also referred hereinafter to as “SC difference”) is large.

Thus, the motion detection unit 1312 can determine that the non-fluid part moves if the SC difference is equal to or higher than a predetermined SC difference threshold. Moreover, the motion detection can be performed for each pixel, each block, or the entire screen. In addition to the determination of presence or absence of motion, the amount of movement of the image capturing target 2 can be calculated on the basis of the SC difference.

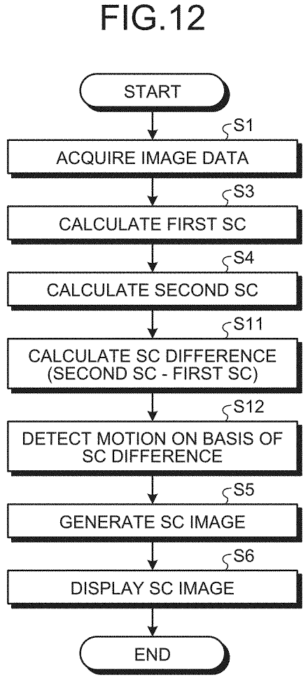

Here, FIG. 12 is a flowchart illustrating the third SC image generation processing performed by the information processing apparatus 13 according to the second embodiment of the present disclosure. The description of the matters similar to the flowchart in FIG. 8 will be omitted as appropriate.

In step S1, the acquisition unit 1311 initially acquires image data. Subsequently, in step S3, the first SC calculation unit 1313 calculates the first SC for each pixel on the basis of the first speckle image. Subsequently, in step S4, the first SC calculation unit 1313 calculates the second SC for each pixel on the basis of the second speckle image.

Subsequently, in step S11, the motion detection unit 1312 calculates, as the SC difference, a value obtained by subtracting the first SC from the second SC.

Subsequently, in step S12, the motion detection unit 1312 detects the motion of the image capturing target 2 on the basis of the SC difference calculated in step S11. Specifically, the motion detection unit 1312 can determine that the image capturing target 2 (non-fluid part) moves if the SC difference is equal to or higher than a predetermined SC difference threshold.

Subsequently, in step S5, the SC image generation unit 1315 generates a SC image on the basis of the first SC and the second SC depending on a result obtained by detecting the motion of the image capturing target 2 in step S12. Specifically, according to the first method of using SC and the second method of using SC as described above, in one example, the SC image generation unit 1315 generates the SC image using the first SC and the second SC on the basis of the presence or absence of motion and the amount of movement of the image capturing target 2. Further, in step S5, the SC image generation unit 1315 discriminates between the blood flow part and the non-blood flow part on the basis of, in one example, the SC image.

Subsequently, in step S6, the display control unit 1316 controls the display apparatus 14 to display the SC image so that the blood flow part and the non-blood flow part can be discriminated on the basis of the SC image generated in step S5. After step S6, the processing ends.

As described above, according to the third SC image generation processing by the information processing apparatus 13 of the second embodiment, it is possible to detect the motion of the image capturing target 2 on the basis of the SC difference. Thus, a satisfactory speckle contrast image can be generated on the basis of the first SC and the second SC depending on the detection result. Moreover, steps S3 and S4 in FIG. 12 are not limited to this order and can be in the reverse order.

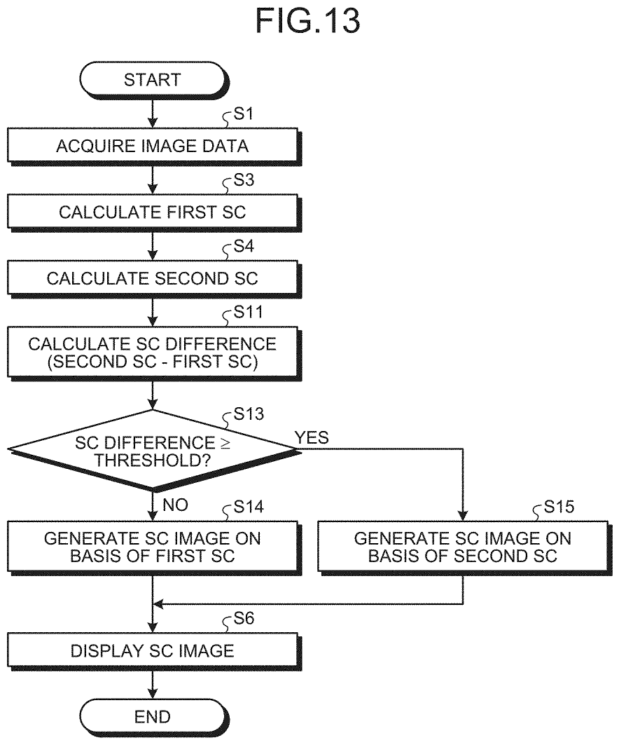

Subsequently, FIG. 13 is a flowchart illustrating the fourth SC image generation processing performed by the information processing apparatus according to the second embodiment of the present disclosure. The description of the matters similar to FIG. 12 will be omitted as appropriate.

In step S1, the acquisition unit 1311 initially acquires image data. Subsequently, in step S3, the first SC calculation unit 1313 calculates the first SC for each pixel on the basis of the first speckle image. Subsequently, in step S4, the first SC calculation unit 1313 calculates the second SC for each pixel on the basis of the second speckle image.

Subsequently, in step S11, the motion detection unit 1312 calculates, as the SC difference, a value obtained by subtracting the first SC from the second SC.

Subsequently, in step S13, the motion detection unit 1312 determines whether or not the SC difference calculated in step S11 is equal to or higher than a predetermined SC difference threshold. If the result is Yes, the processing proceeds to step S15, and if the result is No, the processing proceeds to step S14.

In step S14, the SC image generation unit 1315 generates the speckle contrast image on the basis of the first SC. Further, in step S14, the SC image generation unit 1315 discriminates between the blood flow part and the non-blood flow part on the basis of, in one example, the SC image.

Further, in step S15, the SC image generation unit 1315 generates the speckle contrast image on the basis of the second SC. Further, in step S15, the SC image generation unit 1315 discriminates between the blood flow part and the non-blood flow part on the basis of, in one example, the SC image.

After steps S14 and S15, in step S6, the display control unit 1316 controls the display apparatus 14 to display the SC image so that the blood flow part and the non-blood flow part can be discriminated on the basis of the SC image generated. After step S6, the processing ends.

As described above, according to the fourth SC image generation processing by the information processing apparatus 13 of the second embodiment, it is possible to generate the SC image by calculating only one of the first and second SCs depending on whether or not the SC difference is equal to or higher than a predetermined SC difference threshold, allowing simplifying the processing.

### Third Embodiment

The description is now given of the third embodiment. The same matters as in the first embodiment will be omitted as appropriate.

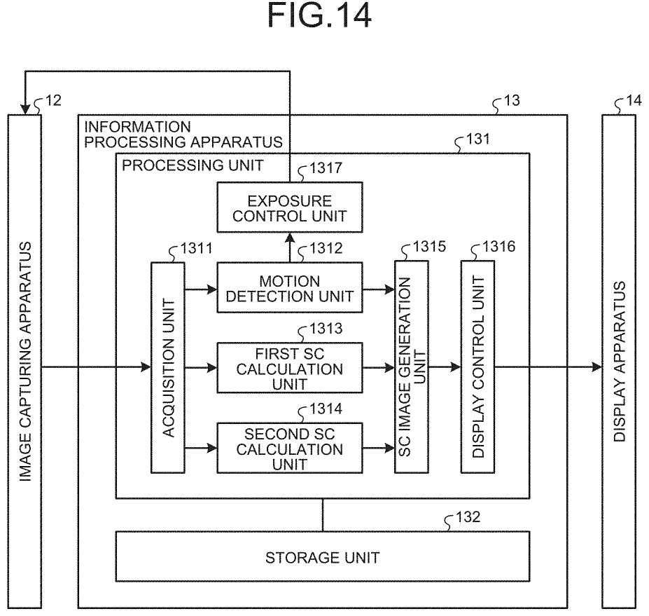

FIG. 14 is a diagram illustrating an exemplary configuration of an information processing apparatus 13 according to the third embodiment of the present disclosure. The information processing apparatus 13 of FIG. 14 is different from the information processing apparatus 13 of FIG. 2 in that the processing unit 131 is additionally provided with an exposure control unit 1317.

The exposure control unit 1317 controls the exposure time of the image capturing apparatus 12 on the basis of the motion of the image capturing target 2 detected by the motion detection unit 1312.

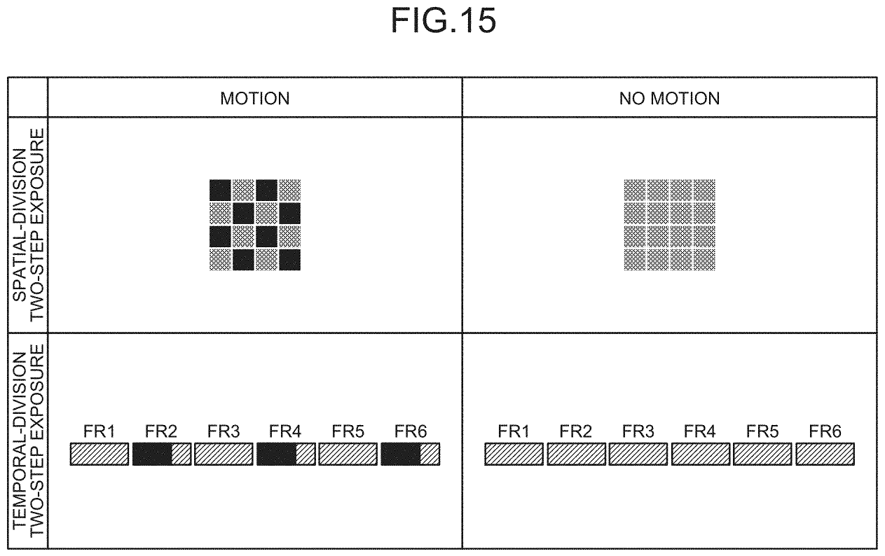

FIG. 15 is herein a diagram illustrated to describe the spatial-division two-step exposure and the temporal-division two-step exposure according to the third embodiment of the present disclosure. To begin with, for the technique of using spatial-division two-step exposure, when the motion detection unit 1312 detects the motion of the image capturing target 2, the exposure control unit 1317 controls the image capturing apparatus 12 to capture a mixed image (FIG. 3). This makes it possible for the first SC calculation unit 1313 to calculate the first SC for each pixel on the basis of the first S pixel in the mixed image and for the second SC calculation unit 1314 to calculate the second SC for each pixel on the basis of the second S pixel in the mixed image.

Further, when the motion detection unit 1312 does not detect the motion of the image capturing target 2, the exposure control unit 1317 controls the image capturing apparatus 12 to capture the first speckle image (FIG. 4). As a result, it is possible for the first SC calculation unit 1313 to calculate the first SC for each pixel on the basis of the first speckle image.

Further, for the technique of using temporal-division two-step exposure, when the motion detection unit 1312 detects the motion of the image capturing target 2, the exposure control unit 1317 controls the image capturing apparatus 12 to alternately capture the first speckle image (frames FR1, FR3, and FR5) and the second speckle image (frames FR2, FR4, and FR6). This makes it possible for the first SC calculation unit 1313 to calculate the first SC for each pixel on the basis of the first speckle image and for the second SC calculation unit 1314 to calculate the second SC for each pixel on the basis of the second speckle image.

Further, when the motion detection unit 1312 does not detect the motion of the image capturing target 2, the exposure control unit 1317 controls the image capturing apparatus 12 to capture the first speckle image (frames FR1 to FR6) only. As a result, it is possible for the first SC calculation unit 1313 to calculate the first SC for each pixel on the basis of the first speckle image.

In this way, the information processing apparatus 13 according to the third embodiment makes it possible to employ only the long-time exposure if there is no motion of the image capturing target 2 that is likely to occupy most of the total time and use both the long-time exposure and the short-time exposure only if there is such motion of the image capturing target 2, allowing simplifying operations or processing.

Further, in one example, for the technique of using temporal-division two-step exposure, the length of the exposure time in the short-time exposure can be variable depending on the amount of movement of the image capturing target 2. In one example, in the case where the amount of movement of the image capturing target 2 is small, the exposure time in the short-time exposure can be one-half of the exposure time in the long-time exposure. In the case where the amount of movement of the image capturing target 2 is large, the exposure time in the short-time exposure can be 1/16 of the exposure time in the long-time exposure. In addition, such time ratios are not limited to ½ or 1/16 and can be ¼, ⅛, or the like. It is possible to reduce or prevent the decrease in SC of the non-fluid part as the exposure time is shortened, but the S/N worsens. Thus, it is only necessary to determine an adequate exposure time depending on the amount of movement of the image capturing target 2.

Further, for the technique of using spatial-division two-step exposure, in the case where there is the motion of the image capturing target 2, all pixels can be an image of the second S pixels rather than a mixed image.

Further, the switching between the case where there is the motion and the case where there is no motion illustrated in FIG. 15 can be performed in block units or screen units.

Further, for the technique of using light-ray split two-step exposure, it is only necessary for the exposure control unit 1317 to change the length and the ratio between lengths of the exposure time of each of the two image capturing apparatuses 12 depending on the amount of movement of the image capturing target 2.

### Application Example 1

The technology according to the present disclosure is applicable to various products. In one example, the technology according to the present disclosure is applicable to an endoscopic surgery system.

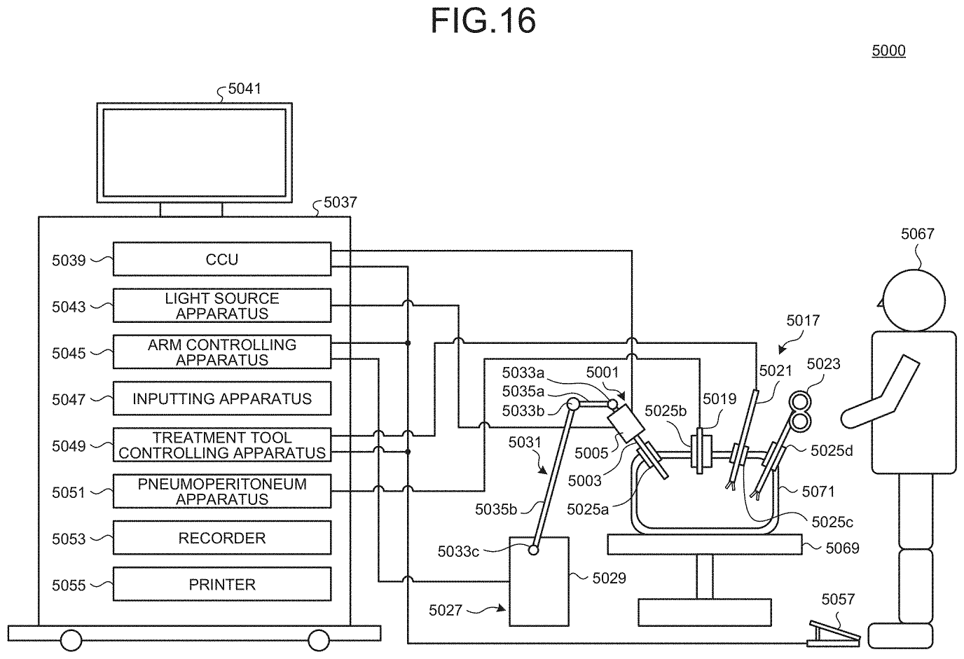

FIG. 16 is a view illustrating an example of a schematic configuration of an endoscopic surgery system 5000 to which the technology according to the present disclosure can be applied. In FIG. 16, a state is illustrated in which a surgeon (medical doctor) 5067 is using the endoscopic surgery system 5000 to perform surgery for a patient 5071 on a patient bed 5069. As illustrated, the endoscopic surgery system 5000 includes an endoscope 5001, other surgical tools 5017, a supporting arm apparatus 5027 which supports the endoscope 5001 thereon, and a cart 5037 on which various apparatus for endoscopic surgery are mounted.

In endoscopic surgery, in place of incision of the abdominal wall to perform laparotomy, a plurality of tubular aperture devices called trocars 5025a to 5025d are used to puncture the abdominal wall. Then, a lens barrel 5003 of the endoscope 5001 and the other surgical tools 5017 are inserted into body cavity of the patient 5071 through the trocars 5025a to 5025d. In the example illustrated, as the other surgical tools 5017, a pneumoperitoneum tube 5019, an energy device 5021 and forceps 5023 are inserted into body cavity of the patient 5071. Further, the energy device 5021 is a treatment tool for performing incision and peeling of a tissue, sealing of a blood vessel or the like by high frequency current or ultrasonic vibration. However, the surgical tools 5017 illustrated are mere examples at all, and as the surgical tools 5017, various surgical tools which are generally used in endoscopic surgery such as, for example, tweezers or a retractor may be used.

An image of a surgical region in a body cavity of the patient 5071 imaged by the endoscope 5001 is displayed on a display apparatus 5041. The surgeon 5067 would use the energy device 5021 or the forceps 5023 while watching the image of the surgical region displayed on the display apparatus 5041 on the real time basis to perform such treatment as, for example, resection of an affected area. It is to be noted that, though not illustrated, the pneumoperitoneum tube 5019, the energy device 5021 and the forceps 5023 are supported by the surgeon 5067, an assistant or the like during surgery.

(Supporting Arm Apparatus)

The supporting arm apparatus 5027 includes an arm unit 5031 extending from a base unit 5029. In the example illustrated, the arm unit 5031 includes joint portions 5033a, 5033b and 5033c and links 5035a and 5035b and is driven under the control of an arm controlling apparatus 5045. The endoscope 5001 is supported by the arm unit 5031 such that the position and the posture of the endoscope 5001 are controlled. Consequently, stable fixation in position of the endoscope 5001 can be implemented.

(Endoscope)

The endoscope 5001 includes the lens barrel 5003 which has a region of a predetermined length from a distal end thereof to be inserted into a body cavity of the patient 5071, and a camera head 5005 connected to a proximal end of the lens barrel 5003. In the example illustrated, the endoscope 5001 is illustrated as a rigid endoscope having the lens barrel 5003 of the hard type. However, the endoscope 5001 may otherwise be configured as a flexible endoscope having the lens barrel 5003 of the flexible type.

The lens barrel 5003 has, at a distal end thereof, an opening in which an objective lens is fitted. A light source apparatus 5043 is connected to the endoscope 5001 such that light generated by the light source apparatus 5043 is introduced to a distal end of the lens barrel by a light guide extending in the inside of the lens barrel 5003 and is irradiated toward an observation target in a body cavity of the patient 5071 through the objective lens. It is to be noted that the endoscope 5001 may be a forward-viewing endoscope or may be an oblique-viewing endoscope or a side-viewing endoscope.

An optical system and an image pickup element are provided in the inside of the camera head 5005 such that reflected light (observation light) from an observation target is condensed on the image pickup element by the optical system. The observation light is photo-electrically converted by the image pickup element to generate an electric signal corresponding to the observation light, namely, an image signal corresponding to an observation image. The image signal is transmitted as RAW data to a CCU 5039. It is to be noted that the camera head 5005 has a function incorporated therein for suitably driving the optical system of the camera head 5005 to adjust the magnification and the focal distance.

It is to be noted that, in order to establish compatibility with, for example, a stereoscopic vision (three dimensional (3D) display), a plurality of image pickup elements may be provided on the camera head 5005. In this case, a plurality of relay optical systems are provided in the inside of the lens barrel 5003 in order to guide observation light to each of the plurality of image pickup elements.

(Various Apparatus Incorporated in Cart)

The CCU 5039 includes a central processing unit (CPU), a graphics processing unit (GPU) or the like and integrally controls operation of the endoscope 5001 and the display apparatus 5041. In particular, the CCU 5039 performs, for an image signal received from the camera head 5005, various image processes for displaying an image based on the image signal such as, for example, a development process (demosaic process). The CCU 5039 provides the image signal for which the image processes have been performed to the display apparatus 5041. Further, the CCU 5039 transmits a control signal to the camera head 5005 to control driving of the camera head 5005. The control signal may include information relating to an image pickup condition such as a magnification or a focal distance.

The display apparatus 5041 displays an image based on an image signal for which the image processes have been performed by the CCU 5039 under the control of the CCU 5039. If the endoscope 5001 is ready for imaging of a high resolution such as 4K (horizontal pixel number 3840×vertical pixel number 2160), 8K (horizontal pixel number 7680×vertical pixel number 4320) or the like and/or ready for 3D display, then a display apparatus by which corresponding display of the high resolution and/or 3D display are possible may be used as the display apparatus 5041. Where the apparatus is ready for imaging of a high resolution such as 4K or 8K, if the display apparatus used as the display apparatus 5041 has a size of equal to or not less than 55 inches, then a more immersive experience can be obtained. Further, a plurality of display apparatus 5041 having different resolutions and/or different sizes may be provided in accordance with purposes.

The light source apparatus 5043 includes a light source such as, for example, a light emitting diode (LED) and supplies irradiation light for imaging of a surgical region to the endoscope 5001.

The arm controlling apparatus 5045 includes a processor such as, for example, a CPU and operates in accordance with a predetermined program to control driving of the arm unit 5031 of the supporting arm apparatus 5027 in accordance with a predetermined controlling method.

An inputting apparatus 5047 is an input interface for the endoscopic surgery system 5000. A user can perform inputting of various kinds of information or instruction inputting to the endoscopic surgery system 5000 through the inputting apparatus 5047. For example, the user would input various kinds of information relating to surgery such as physical information of a patient, information regarding a surgical procedure of the surgery and so forth through the inputting apparatus 5047. Further, the user would input, for example, an instruction to drive the arm unit 5031, an instruction to change an image pickup condition (type of irradiation light, magnification, focal distance or the like) by the endoscope 5001, an instruction to drive the energy device 5021 or the like through the inputting apparatus 5047.

The type of the inputting apparatus 5047 is not limited and may be that of any one of various known inputting apparatus. As the inputting apparatus 5047, for example, a mouse, a keyboard, a touch panel, a switch, a foot switch 5057 and/or a lever or the like may be applied. Where a touch panel is used as the inputting apparatus 5047, it may be provided on the display face of the display apparatus 5041.

Otherwise, the inputting apparatus 5047 is a device to be mounted on a user such as, for example, a glasses type wearable device or a head mounted display (HMD), and various kinds of inputting are performed in response to a gesture or a line of sight of the user detected by any of the devices mentioned. Further, the inputting apparatus 5047 includes a camera which can detect a motion of a user, and various kinds of inputting are performed in response to a gesture or a line of sight of a user detected from a video imaged by the camera. Further, the inputting apparatus 5047 includes a microphone which can collect the voice of a user, and various kinds of inputting are performed by voice collected by the microphone. By configuring the inputting apparatus 5047 such that various kinds of information can be inputted in a contactless fashion in this manner, especially a user who belongs to a clean area (for example, the surgeon 5067) can operate an apparatus belonging to an unclean area in a contactless fashion. Further, since the user can operate an apparatus without releasing a possessed surgical tool from its hand, the convenience to the user is improved.

A treatment tool controlling apparatus 5049 controls driving of the energy device 5021 for cautery or incision of a tissue, sealing of a blood vessel or the like. A pneumoperitoneum apparatus 5051 feeds gas into a body cavity of the patient 5071 through the pneumoperitoneum tube 5019 to inflate the body cavity in order to secure the field of view of the endoscope 5001 and secure the working space for the surgeon. A recorder 5053 is an apparatus capable of recording various kinds of information relating to surgery. A printer 5055 is an apparatus capable of printing various kinds of information relating to surgery in various forms such as a text, an image or a graph.

In the following, especially a characteristic configuration of the endoscopic surgery system 5000 is described in more detail.

(Supporting Arm Apparatus)

The supporting arm apparatus 5027 includes the base unit 5029 serving as a base, and the arm unit 5031 extending from the base unit 5029. In the example illustrated, the arm unit 5031 includes the plurality of joint portions 5033a, 5033b and 5033c and the plurality of links 5035a and 5035b connected to each other by the joint portion 5033b. In FIG. 16, for simplified illustration, the configuration of the arm unit 5031 is illustrated in a simplified form. Actually, the shape, number and arrangement of the joint portions 5033a to 5033c and the links 5035a and 5035b and the direction and so forth of axes of rotation of the joint portions 5033a to 5033c can be set suitably such that the arm unit 5031 has a desired degree of freedom. For example, the arm unit 5031 may preferably be configured such that it has a degree of freedom equal to or not less than 6 degrees of freedom. This makes it possible to move the endoscope 5001 freely within the movable range of the arm unit 5031. Consequently, it becomes possible to insert the lens barrel 5003 of the endoscope 5001 from a desired direction into a body cavity of the patient 5071.

An actuator is provided in each of the joint portions 5033a to 5033c, and the joint portions 5033a to 5033c are configured such that they are rotatable around predetermined axes of rotation thereof by driving of the respective actuators. The driving of the actuators is controlled by the arm controlling apparatus 5045 to control the rotational angle of each of the joint portions 5033a to 5033c thereby to control driving of the arm unit 5031. Consequently, control of the position and the posture of the endoscope 5001 can be implemented. Thereupon, the arm controlling apparatus 5045 can control driving of the arm unit 5031 by various known controlling methods such as force control or position control.

For example, if the surgeon 5067 suitably performs operation inputting through the inputting apparatus 5047 (including the foot switch 5057), then driving of the arm unit 5031 may be controlled suitably by the arm controlling apparatus 5045 in response to the operation input to control the position and the posture of the endoscope 5001. After the endoscope 5001 at the distal end of the arm unit 5031 is moved from an arbitrary position to a different arbitrary position by the control just described, the endoscope 5001 can be supported fixedly at the position after the movement. It is to be noted that the arm unit 5031 may be operated in a master-slave fashion. In this case, the arm unit 5031 may be remotely controlled by the user through the inputting apparatus 5047 which is placed at a place remote from the operating room.

Further, where force control is applied, the arm controlling apparatus 5045 may perform power-assisted control to drive the actuators of the joint portions 5033a to 5033c such that the arm unit 5031 may receive external force by the user and move smoothly following the external force. This makes it possible to move, when the user directly touches with the arm unit 5031 and moves the arm unit 5031, the arm unit 5031 with comparatively weak force. Accordingly, it becomes possible for the user to move the endoscope 5001 more intuitively by a simpler and easier operation, and the convenience to the user can be improved.

Here, generally in endoscopic surgery, the endoscope 5001 is supported by a medical doctor called scopist. In contrast, where the supporting arm apparatus 5027 is used, the position of the endoscope 5001 can be fixed more certainly without hands, and therefore, an image of a surgical region can be obtained stably and surgery can be performed smoothly.

It is to be noted that the arm controlling apparatus 5045 may not necessarily be provided on the cart 5037. Further, the arm controlling apparatus 5045 may not necessarily be a single apparatus. For example, the arm controlling apparatus 5045 may be provided in each of the joint portions 5033a to 5033c of the arm unit 5031 of the supporting arm apparatus 5027 such that the plurality of arm controlling apparatus 5045 cooperate with each other to implement driving control of the and unit 5031.

(Light Source Apparatus)

The light source apparatus 5043 supplies irradiation light upon imaging of a surgical region to the endoscope 5001. The light source apparatus 5043 includes a white light source which includes, for example, an LED, a laser light source or a combination of them. In this case, where a white light source includes a combination of red, green, and blue (RGB) laser light sources, since the output intensity and the output timing can be controlled with a high degree of accuracy for each color (each wavelength), adjustment of the white balance of a picked up image can be performed by the light source apparatus 5043. Further, in this case, if laser beams from the respective RGB laser light sources are irradiated time-divisionally on an observation target and driving of the image pickup elements of the camera head 5005 is controlled in synchronism with the irradiation timings, then images individually corresponding to the R, G and B colors can be picked up time-divisionally. According to the method just described, a color image can be obtained even if a color filter is not provided for the image pickup element.

Further, driving of the light source apparatus 5043 may be controlled such that the intensity of light to be outputted is changed for each predetermined time. By controlling driving of the image pickup element of the camera head 5005 in synchronism with the timing of the change of the intensity of light to acquire images time-divisionally and synthesizing the images, an image of a high dynamic range free from underexposed blocked up shadows and overexposed highlights can be created.

Further, the light source apparatus 5043 may be configured to supply light of a predetermined wavelength band ready for special light observation. In special light observation, for example, by utilizing the wavelength dependency of absorption of light in a body tissue to irradiate light of a narrower wavelength band in comparison with irradiation light upon ordinary observation (namely, white light), narrow band light observation (narrow band imaging) of imaging a predetermined tissue such as a blood vessel of a superficial portion of the mucous membrane or the like in a high contrast is performed. Alternatively, in special light observation, fluorescent observation for obtaining an image from fluorescent light generated by irradiation of excitation light may be performed. In fluorescent observation, it is possible to perform observation of fluorescent light from a body tissue by irradiating excitation light on the body tissue (autofluorescence observation) or to obtain a fluorescent light image by locally injecting a reagent such as indocyanine green (ICG) into a body tissue and irradiating excitation light corresponding to a fluorescent light wavelength of the reagent upon the body tissue. The light source apparatus 5043 can be configured to supply such narrow-band light and/or excitation light suitable for special light observation as described above.

(Camera Head and CCU)

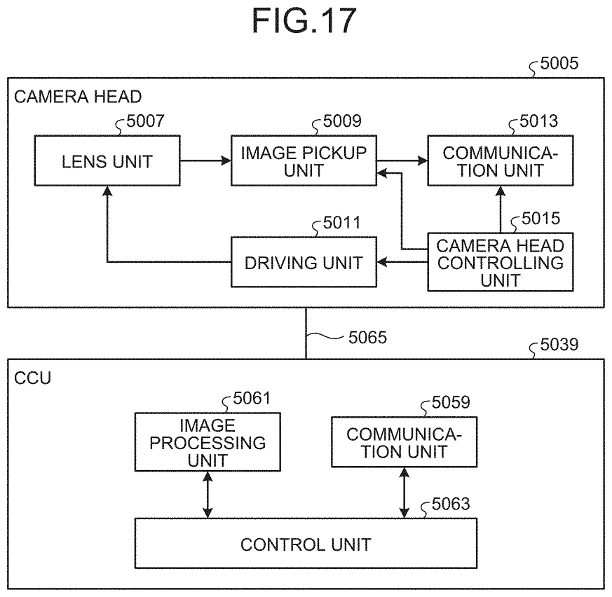

Functions of the camera head 5005 of the endoscope 5001 and the CCU 5039 are described in more detail with reference to FIG. 17. FIG. 17 is a block diagram illustrating an example of a functional configuration of the camera head 5005 and the CCU 5039 illustrated in FIG. 16.

Referring to FIG. 17, the camera head 5005 has, as functions thereof, a lens unit 5007, an image pickup unit 5009, a driving unit 5011, a communication unit 5013 and a camera head controlling unit 5015. Further, the CCU 5039 has, as functions thereof, a communication unit 5059, an image processing unit 5061 and a control unit 5063. The camera head 5005 and the CCU 5039 are connected to be bidirectionally communicable to each other by a transmission cable 5065.

First, a functional configuration of the camera head 5005 is described. The lens unit 5007 is an optical system provided at a connecting location of the camera head 5005 to the lens barrel 5003. Observation light taken in from a distal end of the lens barrel 5003 is introduced into the camera head 5005 and enters the lens unit 5007. The lens unit 5007 includes a combination of a plurality of lenses including a zoom lens and a focusing lens. The lens unit 5007 has optical properties adjusted such that the observation light is condensed on a light receiving face of the image pickup element of the image pickup unit 5009. Further, the zoom lens and the focusing lens are configured such that the positions thereof on their optical axis are movable for adjustment of the magnification and the focal point of a picked up image.

The image pickup unit 5009 includes an image pickup element and disposed at a succeeding stage to the lens unit 5007. Observation light having passed through the lens unit 5007 is condensed on the light receiving face of the image pickup element, and an image signal corresponding to the observation image is generated by photoelectric conversion of the image pickup element. The image signal generated by the image pickup unit 5009 is provided to the communication unit 5013.

As the image pickup element which is included by the image pickup unit 5009, an image sensor, for example, of the complementary metal oxide semiconductor (CMOS) type is used which has a Bayer array and is capable of picking up an image in color. It is to be noted that, as the image pickup element, an image pickup element may be used which is ready, for example, for imaging of an image of a high resolution equal to or not less than 4K. If an image of a surgical region is obtained in a high resolution, then the surgeon 5067 can comprehend a state of the surgical region in enhanced details and can proceed with the surgery more smoothly.

Further, the image pickup element which is included by the image pickup unit 5009 includes such that it has a pair of image pickup elements for acquiring image signals for the right eye and the left eye compatible with 3D display. Where 3D display is applied, the surgeon 5067 can comprehend the depth of a living body tissue in the surgical region more accurately. It is to be noted that, if the image pickup unit 5009 is configured as that of the multi-plate type, then a plurality of systems of lens units 5007 are provided corresponding to the individual image pickup elements of the image pickup unit 5009.

The image pickup unit 5009 may not necessarily be provided on the camera head 5005. For example, the image pickup unit 5009 may be provided just behind the objective lens in the inside of the lens barrel 5003.

The driving unit 5011 includes an actuator and moves the zoom lens and the focusing lens of the lens unit 5007 by a predetermined distance along the optical axis under the control of the camera head controlling unit 5015. Consequently, the magnification and the focal point of a picked up image by the image pickup unit 5009 can be adjusted suitably.

The communication unit 5013 includes a communication apparatus for transmitting and receiving various kinds of information to and from the CCU 5039. The communication unit 5013 transmits an image signal acquired from the image pickup unit 5009 as RAW data to the CCU 5039 through the transmission cable 5065. Thereupon, in order to display a picked up image of a surgical region in low latency, preferably the image signal is transmitted by optical communication. This is because, upon surgery, the surgeon 5067 performs surgery while observing the state of an affected area through a picked up image, it is demanded for a moving image of the surgical region to be displayed on the real time basis as far as possible in order to achieve surgery with a higher degree of safety and certainty. Where optical communication is applied, a photoelectric conversion module for converting an electric signal into an optical signal is provided in the communication unit 5013. After the image signal is converted into an optical signal by the photoelectric conversion module, it is transmitted to the CCU 5039 through the transmission cable 5065.

Further, the communication unit 5013 receives a control signal for controlling driving of the camera head 5005 from the CCU 5039. The control signal includes information relating to image pickup conditions such as, for example, information that a frame rate of a picked up image is designated, information that an exposure value upon image picking up is designated and/or information that a magnification and a focal point of a picked up image are designated. The communication unit 5013 provides the received control signal to the camera head controlling unit 5015. It is to be noted that also the control signal from the CCU 5039 may be transmitted by optical communication. In this case, a photoelectric conversion module for converting an optical signal into an electric signal is provided in the communication unit 5013. After the control signal is converted into an electric signal by the photoelectric conversion module, it is provided to the camera head controlling unit 5015.

It is to be noted that the image pickup conditions such as the frame rate, exposure value, magnification or focal point are set automatically by the control unit 5063 of the CCU 5039 on the basis of an acquired image signal. In other words, an auto exposure (AE) function, an auto focus (AF) function and an auto white balance (AWB) function are incorporated in the endoscope 5001.

The camera head controlling unit 5015 controls driving of the camera head 5005 on the basis of a control signal from the CCU 5039 received through the communication unit 5013. For example, the camera head controlling unit 5015 controls driving of the image pickup element of the image pickup unit 5009 on the basis of information that a frame rate of a picked up image is designated and/or information that an exposure value upon image picking up is designated. Further, for example, the camera head controlling unit 5015 controls the driving unit 5011 to suitably move the zoom lens and the focus lens of the lens unit 5007 on the basis of information that a magnification and a focal point of a picked up image are designated. The camera head controlling unit 5015 may further include a function for storing information for identifying the lens barrel 5003 and/or the camera head 5005.

It is to be noted that, by disposing the components such as the lens unit 5007 and the image pickup unit 5009 in a sealed structure having high airtightness and waterproof, the camera head 5005 can be provided with resistance to an autoclave sterilization process.

Now, a functional configuration of the CCU 5039 is described. The communication unit 5059 includes a communication apparatus for transmitting and receiving various kinds of information to and from the camera head 5005. The communication unit 5059 receives an image signal transmitted thereto from the camera head 5005 through the transmission cable 5065. Thereupon, the image signal may be transmitted preferably by optical communication as described above. In this case, for the compatibility with optical communication, the communication unit 5059 includes a photoelectric conversion module for converting an optical signal into an electric signal. The communication unit 5059 provides the image signal after conversion into an electric signal to the image processing unit 5061.

Further, the communication unit 5059 transmits, to the camera head 5005, a control signal for controlling driving of the camera head 5005. The control signal may also be transmitted by optical communication.

The image processing unit 5061 performs various image processes for an image signal in the form of RAW data transmitted thereto from the camera head 5005. The image processes include various known signal processes such as, for example, a development process, an image quality improving process (a band width enhancement process, a super-resolution process, a noise reduction (NR) process and/or an image stabilization process) and/or an enlargement process (electronic zooming process). Further, the image processing unit 5061 performs a detection process for an image signal in order to perform AE, AF and AWB.

The image processing unit 5061 includes a processor such as a CPU or a GPU, and when the processor operates in accordance with a predetermined program, the image processes and the detection process described above can be performed. It is to be noted that, where the image processing unit 5061 includes a plurality of GPUs, the image processing unit 5061 suitably divides information relating to an image signal such that image processes are performed in parallel by the plurality of GPUs.

The control unit 5063 performs various kinds of control relating to image picking up of a surgical region by the endoscope 5001 and display of the picked up image. For example, the control unit 5063 generates a control signal for controlling driving of the camera head 5005. Thereupon, if image pickup conditions are inputted by the user, then the control unit 5063 generates a control signal on the basis of the input by the user. Alternatively, where the endoscope 5001 has an AE function, an AF function and an AWB function incorporated therein, the control unit 5063 suitably calculates an optimum exposure value, focal distance and white balance in response to a result of a detection process by the image processing unit 5061 and generates a control signal.

Further, the control unit 5063 controls the display apparatus 5041 to display an image of a surgical region on the basis of an image signal for which image processes have been performed by the image processing unit 5061. Thereupon, the control unit 5063 recognizes various objects in the surgical region image using various image recognition technologies. For example, the control unit 5063 can recognize a surgical tool such as forceps, a particular living body region, bleeding, mist when the energy device 5021 is used and so forth by detecting the shape, color and so forth of edges of the objects included in the surgical region image. The control unit 5063 causes, when it controls the display apparatus 5041 to display a surgical region image, various kinds of surgery supporting information to be displayed in an overlapping manner with an image of the surgical region using a result of the recognition. Where surgery supporting information is displayed in an overlapping manner and presented to the surgeon 5067, the surgeon 5067 can proceed with the surgery more safety and certainty.

The transmission cable 5065 which connects the camera head 5005 and the CCU 5039 to each other is an electric signal cable ready for communication of an electric signal, an optical fiber ready for optical communication or a composite cable ready for both of electrical and optical communication.

Here, while, in the example illustrated, communication is performed by wired communication using the transmission cable 5065, the communication between the camera head 5005 and the CCU 5039 may be performed otherwise by wireless communication. Where the communication between the camera head 5005 and the CCU 5039 is performed by wireless communication, there is no necessity to lay the transmission cable 5065 in the operating room. Therefore, such a situation that movement of medical staff in the operating room is disturbed by the transmission cable 5065 can be eliminated.

An example of the endoscopic surgery system 5000 to which the technology according to an embodiment of the present disclosure can be applied has been described above. It is to be noted here that, although the endoscopic surgery system 5000 has been described as an example, the system to which the technology according to an embodiment of the present disclosure can be applied is not limited to the example. For example, the technology according to an embodiment of the present disclosure may be applied to a flexible endoscopic surgery system for inspection or a microscopic surgery system that will be described in application example 2 below,

The technology according to the present disclosure is suitably applicable to the endoscope 5001 among the configurations described above. Specifically, in the case where the blood flow part and the non-blood flow part in the image of the surgical site in the body cavity of the patient 5071 captured by the endoscope 5001 are displayed in a visibly recognizable manner on the display apparatus 5041 with ease, the technology according to the present disclosure is applicable. The technology according to the present disclosure applied to the endoscope 5001 allows for the generation of a satisfactory SC image that is accurately discriminated between the blood flow and non-blood flow parts even in the case where the captured image moves. This makes it possible for the surgeon 5067 to achieve real-time viewing of the image of the surgical site in which the blood flow and non-blood flow parts are accurately discriminated through the display apparatus 5041, leading to safer surgery.

### Application Example 2

Further, the technology according to the present disclosure may be applied to a microscopic surgery system used for so-called microsurgery that is performed while enlarging a minute region of a patient for observation.

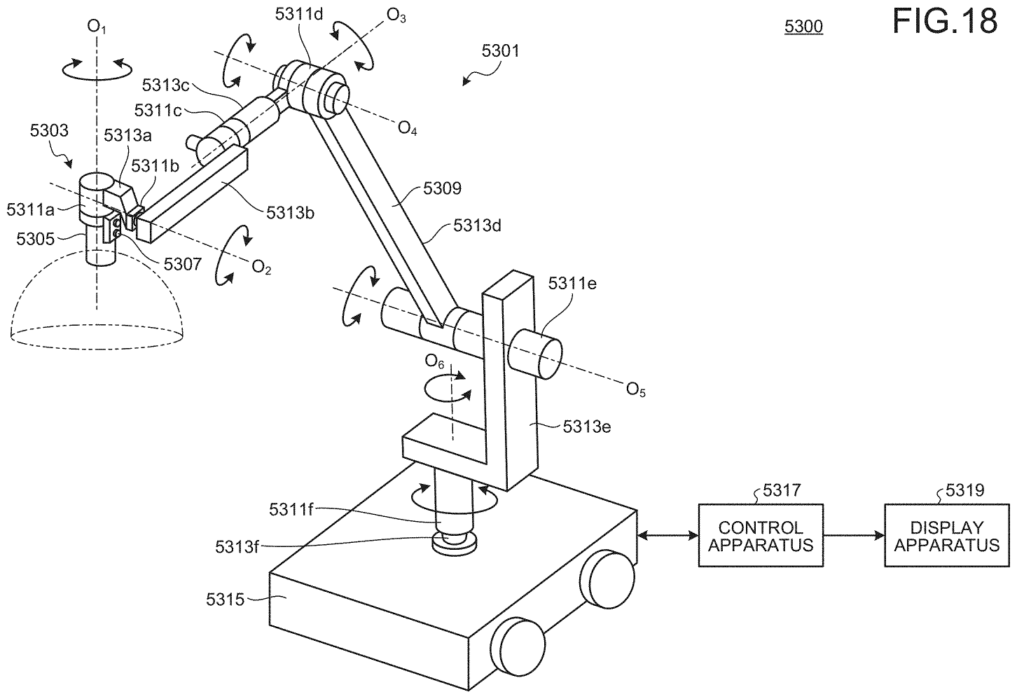

FIG. 18 is a view illustrating an example of a schematic configuration of a microscopic surgery system 5300 to which the technology according to the present disclosure can be applied. Referring to FIG. 18, the microscopic surgery system 5300 includes a microscope apparatus 5301, a control apparatus 5317 and a display apparatus 5319. It is to be noted that, in the description of the microscopic surgery system 5300, the term “user” signifies an arbitrary one of medical staff members such as a surgery or an assistant who uses the microscopic surgery system 5300.

The Microscope apparatus 5301 has a microscope unit 5303 for enlarging an observation target (surgical region of a patient) for observation, an arm unit 5309 which supports the microscope unit 5303 at a distal end thereof, and a base unit 5315 which supports a proximal end of the arm unit 5309.

The microscope unit 5303 includes a cylindrical portion 5305 of a substantially cylindrical shape, an image pickup unit (not illustrated) provided in the inside of the cylindrical portion 5305, and an operation unit 5307 provided in a partial region of an outer circumference of the cylindrical portion 5305. The microscope unit 5303 is a microscope unit of the electronic image pickup type (microscope unit of the video type) which picks up an image electronically by the image pickup unit.

A cover glass member for protecting the internal image pickup unit is provided at an opening face of a lower end of the cylindrical portion 5305. Light from an observation target (hereinafter referred to also as observation light) passes through the cover glass member and enters the image pickup unit in the inside of the cylindrical portion 5305. It is to be noted that a light source includes, for example, a light emitting diode (LED) or the like may be provided in the inside of the cylindrical portion 5305, and upon image picking up, light may be irradiated upon an observation target from the light source through the cover glass member.

The image pickup unit includes an optical system which condenses observation light, and an image pickup element which receives the observation light condensed by the optical system. The optical system includes a combination of a plurality of lenses including a zoom lens and a focusing lens. The optical system has optical properties adjusted such that the observation light is condensed to be formed image on a light receiving face of the image pickup element. The image pickup element receives and photoelectrically converts the observation light to generate a signal corresponding to the observation light, namely, an image signal corresponding to an observation image. As the image pickup element, for example, an image pickup element which has a Bayer array and is capable of picking up an image in color is used. The image pickup element may be any of various known image pickup elements such as a complementary metal oxide semiconductor (CMOS) image sensor or a charge coupled device (CCD) image sensor. The image signal generated by the image pickup element is transmitted as RAW data to the control apparatus 5317. Here, the transmission of the image signal may be performed suitably by optical communication. This is because, since, at a surgery site, the surgeon performs surgery while observing the state of an affected area through a picked up image, in order to achieve surgery with a higher degree of safety and certainty, it is demanded for a moving image of the surgical region to be displayed on the real time basis as far as possible. Where optical communication is used to transmit the image signal, the picked up image can be displayed with low latency.

It is to be noted that the image pickup unit may have a driving mechanism for moving the zoom lens and the focusing lens of the optical system thereof along the optical axis. Where the zoom lens and the focusing lens are moved suitably by the driving mechanism, the magnification of the picked up image and the focal distance upon image picking up can be adjusted. Further, the image pickup unit may incorporate therein various functions which may be provided generally in a microscopic unit of the electronic image pickup such as an auto exposure (AE) function or an auto focus (AF) function.

Further the image pickup unit may be configured as an image pickup unit of the single-plate type which includes a single image pickup element or may be configured as an image pickup unit of the multi-plate type which includes a plurality of image pickup elements. Where the image pickup unit is configured as that of the multi-plate type, for example, image signals corresponding to red, green, and blue colors may be generated by the image pickup elements and may be synthesized to obtain a color image. Alternatively, the image pickup unit may be configured such that it has a pair of image pickup elements for acquiring image signals for the right eye and the left eye compatible with a stereoscopic vision (three dimensional (3D) display). Where 3D display is applied, the surgeon can comprehend the depth of a living body tissue in the surgical region with a higher degree of accuracy. It is to be noted that, if the image pickup unit is configured as that of stereoscopic type, then a plurality of optical systems are provided corresponding to the individual image pickup elements.

The operation unit 5307 includes, for example, a cross lever, a switch or the like and accepts an operation input of the user. For example, the user can input an instruction to change the magnification of the observation image and the focal distance to the observation target through the operation unit 5307. The magnification and the focal distance can be adjusted by the driving mechanism of the image pickup unit suitably moving the zoom lens and the focusing lens in accordance with the instruction. Further, for example, the user can input an instruction to switch the operation mode of the arm unit 5309 (an all-free mode and a fixed mode hereinafter described) through the operation unit 5307. It is to be noted that when the user intends to move the microscope unit 5303, it is supposed that the user moves the microscope unit 5303 in a state in which the user grasps the microscope unit 5303 holding the cylindrical portion 5305. Accordingly, the operation unit 5307 is preferably provided at a position at which it can be operated readily by the fingers of the user with the cylindrical portion 5305 held such that the operation unit 5307 can be operated even while the user is moving the cylindrical portion 5305.

The arm unit 5309 is configured such that a plurality of links (first link 5313a to sixth link 5313f) are connected for rotation relative to each other by a plurality of joint portions (first joint portion 5311a to sixth joint portion 5311f).

The first joint portion 5311a has a substantially columnar shape and supports, at a distal end (lower end) thereof, an upper end of the cylindrical portion 5305 of the microscope unit 5303 for rotation around an axis of rotation (first axis O1) parallel to the center axis of the cylindrical portion 5305. Here, the first joint portion 5311a may be configured such that the first axis O1 thereof is in alignment with the optical axis of the image pickup unit of the microscope unit 5303. By the configuration, if the microscope unit 5303 is rotated around the first axis O1, then the field of view can be changed so as to rotate the picked up image.

The first link 5313a fixedly supports, at a distal end thereof, the first joint portion 5311a. Specifically, the first link 5313a is a bar-like member having a substantially L shape and is connected to the first joint portion 5311a such that one side at the distal end side thereof extends in a direction orthogonal to the first axis O1 and an end portion of the one side abuts with an upper end portion of an outer periphery of the first joint portion 5311a. The second joint portion 5311b is connected to an end portion of the other side on the proximal end side of the substantially L shape of the first link 5313a.

The second joint portion 5311b has a substantially columnar shape and supports, at a distal end thereof, a proximal end of the first link 5313a for rotation around an axis of rotation (second axis O2) orthogonal to the first axis O1. The second link 5313h is fixedly connected at a distal end thereof to a proximal end of the second joint portion 5311b.

The second link 5313b is a bar-like member having a substantially L shape, and one side of a distal end side of the second link 5313b extends in a direction orthogonal to the second axis O2 and an end portion of the one side is fixedly connected to a proximal end of the second joint portion 5311b. The third joint portion 5311c is connected to the other side at the proximal end side of the substantially L shape of the second link 5313b.

The third joint portion 5311c has a substantially columnar shape and supports, at a distal end thereof, a proximal end of the second link 5313b for rotation around an axis of rotation (third axis O3) orthogonal to the first axis O1 and the second axis O2. The third link 5313c is fixedly connected at a distal end thereof to a proximal end of the third joint portion 5311c. By rotating the components at the distal end side including the microscope unit 5303 around the second axis O2 and the third axis O3, the microscope unit 5303 can be moved such that the position of the microscope unit 5303 is changed within a horizontal plane. In other words, by controlling the rotation around the second axis O2 and the third axis O3, the field of view of the picked up image can be moved within a plane.

The third link 5313c is configured such that the distal end side thereof has a substantially columnar shape, and a proximal end of the third joint portion 5311c is fixedly connected to the distal end of the columnar shape such that both of them have a substantially same center axis. The proximal end side of the third link 5313c has a prismatic shape, and the fourth joint portion 5311d is connected to an end portion of the third link 5313c.

The fourth joint portion 5311d has a substantially columnar shape and supports, at a distal end thereof, a proximal end of the third link 5313c for rotation around an axis of rotation (fourth axis O4) orthogonal to the third axis O3. The fourth link 5313d is fixedly connected at a distal end thereof to a proximal end of the fourth joint portion 5311d.

The fourth link 5313d is a bar-like member extending substantially linearly and is fixedly connected to the fourth joint portion 5311d such that it extends orthogonally to the fourth axis O4 and abuts at an end portion of the distal end thereof with a side face of the substantially columnar shape of the fourth joint portion 5311d. The fifth joint portion 5311e is connected to a proximal end of the fourth link 5313d.

The fifth joint portion 5311e has a substantially columnar shape and supports, at a distal end side thereof, a proximal end of the fourth link 5313d for rotation around an axis of rotation (fifth axis O5) parallel to the fourth axis O4. The fifth link 5313e is fixedly connected at a distal end thereof to a proximal end of the fifth joint portion 5311e. The fourth axis O4 and the fifth axis O5 are axes of rotation around which the microscope unit 5303 can be moved in the upward and downward direction. By rotating the components at the distal end side including the microscope unit 5303 around the fourth axis O4 and the fifth axis O5, the height of the microscope unit 5303, namely, the distance between the microscope unit 5303 and an observation target, can be adjusted.

The fifth link 5313e includes a combination of a first member having a substantially L shape one side of which extends in the vertical direction and the other side of which extends in the horizontal direction, and a bar-like second member extending vertically downwardly from the portion of the first member which extends in the horizontal direction. The fifth joint portion 5311e is fixedly connected at a proximal end thereof to a neighboring upper end of a part extending the first member of the fifth link 5313e in the vertical direction. The sixth joint portion 5311f is connected to proximal end (lower end) of the second member of the fifth link 5313e.

The sixth joint portion 5311f has a substantially columnar shape and supports, at a distal end side thereof, a proximal end of the fifth link 5313e for rotation around an axis of rotation (sixth axis O6) parallel to the vertical direction. The sixth link 5313f is fixedly connected at a distal end thereof to a proximal end of the sixth joint portion 5311f.

The sixth link 5313f is a bar-like member extending in the vertical direction and is fixedly connected at a proximal end thereof to an upper face of the base unit 5315.

The first joint portion 5311a to sixth joint portion 5311f have movable ranges suitably set such that the microscope unit 5303 can make a desired movement. Consequently, in the arm unit 5309 having the configuration described above, a movement of totaling six degrees of freedom including three degrees of freedom for translation and three degrees of freedom for rotation can be implemented with regard to a movement of the microscope unit 5303. By configuring the arm unit 5309 such that six degrees of freedom are implemented for movements of the microscope unit 5303 in this manner, the position and the posture of the microscope unit 5303 can be controlled freely within the movable range of the arm unit 5309. Accordingly, it is possible to observe a surgical region from every angle, and surgery can be executed more smoothly.

It is to be noted that the configuration of the arm unit 5309 as illustrated is an example at all, and the number and shape (length) of the links including the arm unit 5309 and the number, location, direction of the axis of rotation and so forth of the joint portions may be designed suitably such that desired degrees of freedom can be implemented. For example, in order to freely move the microscope unit 5303, preferably the arm unit 5309 is configured so as to have six degrees of freedom as described above. However, the arm unit 5309 may also be configured so as to have much greater degree of freedom (namely, redundant degree of freedom). Where a redundant degree of freedom exists, in the arm unit 5309, it is possible to change the posture of the arm unit 5309 in a state in which the position and the posture of the microscope unit 5303 are fixed. Accordingly, control can be implemented which is higher in convenience to the surgeon such as to control the posture of the arm unit 5309 such that, for example, the arm unit 5309 does not interfere with the field of view of the surgeon who watches the display apparatus 5319.

Here, an actuator in which a driving mechanism such as a motor, an encoder which detects an angle of rotation at each joint portion and so forth are incorporated may be provided for each of the first joint portion 5311a to sixth joint portion 5311f. By suitably controlling driving of the actuators provided in the first joint portion 5311a to sixth joint portion 5311f by the control apparatus 5317, the posture of the arm unit 5309, namely, the position and the posture of the microscope unit 5303, can be controlled. Specifically, the control apparatus 5317 can comprehend the posture of the arm unit 5309 at present and the position and the posture of the microscope unit 5303 at present on the basis of information regarding the angle of rotation of the joint portions detected by the encoders. The control apparatus 5317 uses the comprehended information to calculate a control value (for example, an angle of rotation or torque to be generated) for each joint portion with which a movement of the microscope unit 5303 in accordance with an operation input from the user is implemented. Accordingly, the control apparatus 5317 drives the driving mechanism of each joint portion in accordance with the control value. It is to be noted that, in this case, the control method of the arm unit 5309 by the control apparatus 5317 is not limited, and various known control methods such as force control or position control may be applied.

For example, when the surgeon performs operation inputting suitably through an inputting apparatus not illustrated, driving of the arm unit 5309 may be controlled suitably in response to the operation input by the control apparatus 5317 to control the position and the posture of the microscope unit 5303. By this control, it is possible to support, after the microscope unit 5303 is moved from an arbitrary position to a different arbitrary position, the microscope unit 5303 fixedly at the position after the movement. It is to be noted that, as the inputting apparatus, preferably an inputting apparatus is applied which can be operated by the surgeon even if the surgeon has a surgical tool in its hand such as, for example, a foot switch taking the convenience to the surgeon into consideration. Further, operation inputting may be performed in a contactless fashion on the basis of gesture detection or line-of-sight detection in which a wearable device or a camera which is provided in the operating room is used. This makes it possible even for a user who belongs to a clean area to operate an apparatus belonging to an unclean area with a high degree of freedom. In addition, the arm unit 5309 may be operated in a master-slave fashion. In this case, the arm unit 5309 may be remotely controlled by the user through an inputting apparatus which is placed at a place remote from the operating room.

Further, where force control is applied, the control apparatus 5317 may perform power-assisted control to drive the actuators of the first joint portion 5311a to sixth joint portion 5311f such that the arm unit 5309 may receive external force by the user and move smoothly following the external force. This makes it possible to move, when the user holds and directly moves the position of the microscope unit 5303, the microscope unit 5303 with comparatively weak force. Accordingly, it becomes possible for the user to move the microscope unit 5303 more intuitively by a simpler and easier operation, and the convenience to the user can be improved.

Further, driving of the arm unit 5309 may be controlled such that the arm unit 5309 performs a pivot movement. The pivot movement here is a motion for moving the microscope unit 5303 such that the direction of the optical axis of the microscope unit 5303 is kept toward a predetermined point (hereinafter referred to as pivot point) in a space. Since the pivot movement makes it possible to observe the same observation position from various directions, more detailed observation of an affected area becomes possible. It is to be noted that, where the microscope unit 5303 is configured such that the focal distance thereof is fixed, preferably the pivot movement is performed in a state in which the distance between the microscope unit 5303 and the pivot point is fixed. In this case, it is sufficient if the distance between the microscope unit 5303 and the pivot point is adjusted to a fixed focal distance of the microscope unit 5303 in advance. By the configuration just described, the microscope unit 5303 comes to move on a hemispherical plane (schematically illustrated in FIG. 18) having a radius corresponding to the focal distance centered at the pivot point, and even if the observation direction is changed, a clear captured image can be obtained. On the other hand, where the microscope unit 5303 is configured such that the focal distance thereof is adjustable, the pivot movement may be performed in a state in which the distance between the microscope unit 5303 and the pivot point is variable. In this case, for example, the control apparatus 5317 may calculate the distance between the microscope unit 5303 and the pivot point on the basis of information regarding the angles of rotation of the joint portions detected by the encoders and automatically adjust the focal distance of the microscope unit 5303 on the basis of a result of the calculation. Alternatively, where the microscope unit 5303 includes an AF function, adjustment of the focal distance may be performed automatically by the AF function every time the changing in distance caused by the pivot movement between the microscope unit 5303 and the pivot point.

Further, each of the first joint portion 5311a to sixth joint portion 5311f may be provided with a brake for constraining the rotation of the first joint portion 5311a to sixth joint portion 5311f. Operation of the brake may be controlled by the control apparatus 5317. For example, if it is intended to fix the position and the posture of the microscope unit 5303, then the control apparatus 5317 renders the brakes of the joint portions operative. Consequently, even if the actuators are not driven, the posture of the arm unit 5309, namely, the position and posture of the microscope unit 5303, can be fixed, and therefore, the power consumption can be reduced. When it is intended to move the position and the posture of the microscope unit 5303, it is sufficient if the control apparatus 5317 releases the brakes of the joint portions and drives the actuators in accordance with a predetermined control method.

Such operation of the brakes may be performed in response to an operation input by the user through the operation unit 5307 described hereinabove. When the user intends to move the position and the posture of the microscope unit 5303, the user would operate the operation unit 5307 to release the brakes of the joint portions. Consequently, the operation mode of the arm unit 5309 changes to a mode in which rotation of the joint portions can be performed freely (all-free mode). On the other hand, if the user intends to fix the position and the posture of the microscope unit 5303, then the user would operate the operation unit 5307 to render the brakes of the joint portions operative. Consequently, the operation mode of the arm unit 5309 changes to a mode in which rotation of the joint portions is constrained (fixed mode).

The control apparatus 5317 integrally controls operation of the microscopic surgery system 5300 by controlling operation of the microscope apparatus 5301 and the display apparatus 5319. For example, the control apparatus 5317 renders the actuators of the first joint portion 5311a to sixth joint portion 5311f operative in accordance with a predetermined control method to control driving of the arm unit 5309. Further, for example, the control apparatus 5317 controls operation of the brakes of the first joint portion 5311a to sixth joint portion 5311f to change the operation mode of the arm unit 5309. Further, for example, the control apparatus 5317 performs various signal processes for an image signal acquired by the image pickup unit of the microscope unit 5303 of the microscope apparatus 5301 to generate image data for display and controls the display apparatus 5319 to display the generated image data. As the signal processes, various known signal processes such as, for example, a development process (demosaic process), an image quality improving process (a bandwidth enhancement process, a super-resolution process, a noise reduction (NR) process and/or an image stabilization process) and/or an enlargement process (namely, an electronic zooming process) may be performed.

It is to be noted that communication between the control apparatus 5317 and the microscope unit 5303 and communication between the control apparatus 5317 and the first joint portion 5311a to sixth joint portion 5311f may be wired communication or wireless communication. Where wired communication is applied, communication by an electric signal may be performed or optical communication may be performed. In this case, a cable for transmission used for wired communication may be configured as an electric signal cable, an optical fiber or a composite cable of them in response to an applied communication method. On the other hand, where wireless communication is applied, since there is no necessity to lay a transmission cable in the operating room, such a situation that movement of medical staff in the operating room is disturbed by a transmission cable can be eliminated.

The control apparatus 5317 may be a processor such as a central processing unit (CPU) or a graphics processing unit (GPU), or a microcomputer or a control board in which a processor and a storage element such as a memory are incorporated. The various functions described hereinabove can be implemented by the processor of the control apparatus 5317 operating in accordance with a predetermined program. It is to be noted that, in the example illustrated, the control apparatus 5317 is provided as an apparatus separate from the microscope apparatus 5301. However, the control apparatus 5317 may be installed in the inside of the base unit 5315 of the microscope apparatus 5301 and configured integrally with the microscope apparatus 5301. The control apparatus 5317 may also include a plurality of apparatus. For example, microcomputers, control boards or the like may be disposed in the microscope unit 5303 and the first joint portion 5311a to sixth joint portion 5311f of the arm unit 5309 and connected for communication with each other to implement functions similar to those of the control apparatus 5317.

The display apparatus 5319 is provided in the operating room and displays an image corresponding to image data generated by the control apparatus 5317 under the control of the control apparatus 5317. In other words, an image of a surgical region picked up by the microscope unit 5303 is displayed on the display apparatus 5319. The display apparatus 5319 may display, in place of or in addition to an image of a surgical region, various kinds of information relating to the surgery such as physical information of a patient or information regarding a surgical procedure of the surgery. In this case, the display of the display apparatus 5319 may be switched suitably in response to an operation by the user. Alternatively, a plurality of such display apparatus 5319 may also be provided such that an image of a surgical region or various kinds of information relating to the surgery may individually be displayed on the plurality of display apparatus 5319. It is to be noted that, as the display apparatus 5319, various known display apparatus such as a liquid crystal display apparatus or an electro luminescence (EL) display apparatus may be applied.

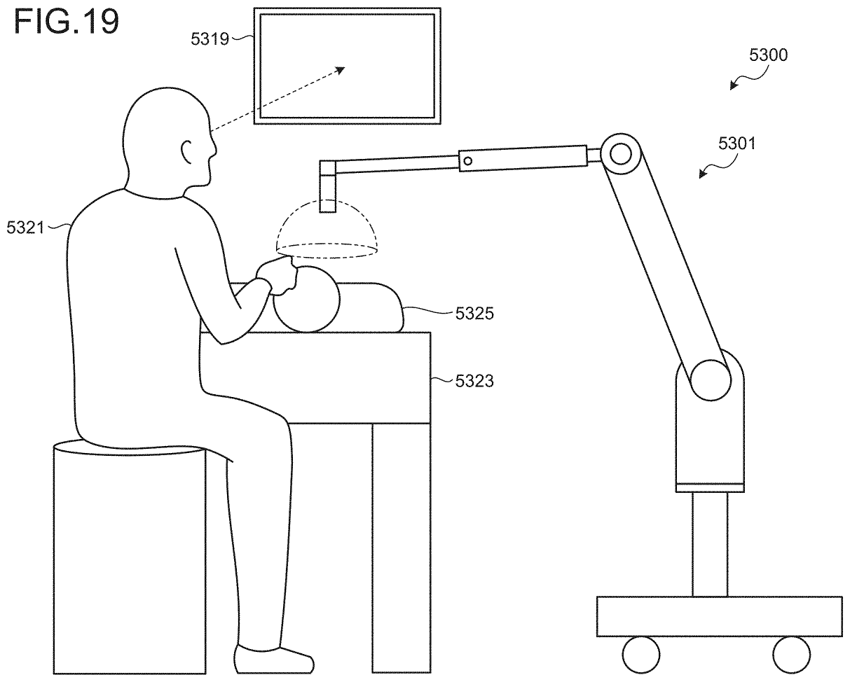

FIG. 19 is a view illustrating a state of surgery in which the microscopic surgery system 5300 illustrated in FIG. 18 is used. FIG. 19 schematically illustrates a state in which a surgeon 5321 uses the microscopic surgery system 5300 to perform surgery for a patient 5325 on a patient bed 5323. It is to be noted that, in FIG. 19, for simplified illustration, the control apparatus 5317 from among the components of the microscopic surgery system 5300 is omitted and the microscope apparatus 5301 is illustrated in a simplified form.

As illustrated in FIG. 2C, upon surgery, using the microscopic surgery system 5300, an image of a surgical region picked up by the microscope apparatus 5301 is displayed in an enlarged scale on the display apparatus 5319 installed on a wall face of the operating room. The display apparatus 5319 is installed at a position opposing to the surgeon 5321, and the surgeon 5321 would perform various treatments for the surgical region such as, for example, resection of the affected area while observing a state of the surgical region from a video displayed on the display apparatus 5319.

An example of the microscopic surgery system 5300 to which the technology according to an embodiment of the present disclosure can be applied has been described. It is to be noted here that, while the microscopic surgery system 5300 is described as an example, the system to which the technology according to an embodiment of the present disclosure can be applied is not limited to this example. For example, the microscope apparatus 5301 may also function as a supporting arm apparatus which supports, at a distal end thereof, a different observation apparatus or some other surgical tool in place of the microscope unit 5303. As the other observation apparatus, for example, an endoscope may be applied. Further, as the different surgical tool, forceps, tweezers, a pneumoperitoneum tube for pneumoperitoneum or an energy device for performing incision of a tissue or sealing of a blood vessel by cautery and so forth can be applied. By supporting any of such an observation apparatus and surgical tools as just described by the supporting apparatus, the position of them can be fixed with a high degree of stability in comparison with that in an alternative case in which they are supported by hands of medical staff. Accordingly, the burden on the medical staff can be reduced. The technology according to an embodiment of the present disclosure may be applied to a supporting arm apparatus which supports such a component as described above other than the microscopic unit.

The technology according to the present disclosure is suitably applicable to the control apparatus 5317 among the configurations described above. Specifically, in the case where the blood flow part and the non-blood flow part in the image of the surgical site of the patient 5325 captured by the image pickup unit of the microscope unit 5303 are displayed in a visibly recognizable manner on the display apparatus 5319 with ease, the technology according to the present disclosure is applicable. The technology according to the present disclosure applied to the control apparatus 5317 allows for the generation of a satisfactory SC image that is accurately discriminated between the blood flow and non-blood flow parts even in the case where the captured image moves. This makes it possible for the surgeon 5321 to achieve real-time viewing of the image of the surgical site in which the blood flow and non-blood flow parts are accurately discriminated through the display apparatus 5319, leading to safer surgery.

Note that the present technology may include the following configuration.

(1) A medical system comprising:

light irradiation means for irradiating an image capturing target with coherent light;

image capturing means for capturing a speckle image obtained from scattered light caused by the image capturing target irradiated with the coherent light;

acquisition means for acquiring a first speckle image at a first exposure time and a second speckle image at a second exposure time shorter than the first exposure time;

speckle contrast calculation means for calculating a first speckle contrast value for each pixel based on the first speckle image and/or a second speckle contrast value for each pixel based on the second speckle image;

motion detection means for detecting motion of the image capturing target; and

speckle image generation means for generating a speckle contrast image on a basis of the first speckle contrast value and/or the second speckle contrast value depending on a detection result of the motion of the image capturing target by the motion detection means.

(2) The medical system according to (1), wherein the medical system is a microscopic surgery system or an endoscopic surgery system.
(3) An information processing apparatus comprising:

acquisition means for acquiring a first speckle image at a first exposure time and a second speckle image at a second exposure time as a speckle image obtained from scattered light caused by an image capturing target irradiated with coherent light, the second exposure time being shorter than the first exposure time;

motion detection means for detecting motion of the image capturing target;

speckle contrast calculation means for calculating a first speckle contrast value for each pixel based on the first speckle image and/or a second speckle contrast value for each pixel based on the second speckle image; and

speckle image generation means for generating a speckle contrast image on a basis of the first speckle contrast value and/or the second speckle contrast value depending on a detection result of the motion of the image capturing target by the motion detection means.

(4) The information processing apparatus according to (3), wherein

the acquisition means acquires a mixed image including a pixel of the first speckle image and a pixel of the second speckle image in one frame, and

the speckle contrast calculation means calculates the first speckle contrast value for each pixel on a basis of the pixel of the first speckle image in the mixed image and calculates the second speckle contrast value for each pixel on a basis of the pixel of the second speckle image in the mixed image.

(5) The information processing apparatus according to (3), wherein the acquisition means alternately acquires the first speckle image and the second speckle image in a time-series order.
(6) The information processing apparatus according to (3), wherein the acquisition means acquires the first speckle image and the second speckle image respectively from two image capturing means having different exposure time periods.
   (7) The information processing apparatus according to (3), wherein
the acquisition means acquires a high frame rate speckle image, and
the speckle contrast calculation means calculates the first speckle contrast value by using a plurality of frames of the high frame rate speckle image as the first speckle image, and
calculates the second speckle contrast value by using one frame of the high frame rate speckle image as the second speckle image.
   (8) The information processing apparatus according to any of (3) to (7), wherein

the speckle image generation means

generates the speckle contrast image on a basis of the first speckle contrast value upon no detection of the motion of the image capturing target by the motion detection means, and

generates the speckle contrast image on a basis of the second speckle contrast value upon detection of the motion of the image capturing target by the motion detection means.

(9) The information processing apparatus according to any of (3) to (7), wherein

the speckle image generation means

generates the speckle contrast image by using a speckle contrast value obtained by weighting and summing and synthesizing the first speckle contrast value and the second speckle contrast value on a basis of an amount of movement of the image capturing target detected by the motion detection means.

(10) The information processing apparatus according to any of (3) to (9), wherein

the motion detection means

detects the motion of the image capturing target on a basis of a value obtained by subtracting the first speckle contrast value from the second speckle contrast value.

(11). The information processing apparatus according to any of (3) to (10), further comprising: exposure control means for controlling an exposure time of image capturing means for capturing the speckle image on a basis of the motion of the image capturing target detected by the motion detection means.
(12) An information processing method comprising:

an acquisition process of acquiring a first speckle image at a first exposure time and a second speckle image at a second exposure time as a speckle image obtained from scattered light caused by an image capturing target irradiated with coherent light, the second exposure time being shorter than the first exposure time;

a motion detection process of detecting motion of the image capturing target;

a speckle contrast calculation process of calculating a first speckle contrast value for each pixel based on the first speckle image and/or a second speckle contrast value for each pixel based on the second speckle image; and

a speckle image generation process of generating a speckle contrast image on a basis of the first speckle contrast value and/or the second speckle contrast value depending on a detection result of the motion of the image capturing target in the motion detection process.

Although the description above is give of the embodiments and modifications of the present disclosure, the technical scope of the present disclosure is not limited to the above-described embodiments and modifications as they are, and various modifications and variations can be made without departing from the spirit and scope of the present disclosure. In addition, components covering different embodiments and modifications can be combined as appropriate.

In one example, the information processing apparatus 13 according to the second embodiment can be additionally provided with the exposure control unit 1317, similar to the case where the information processing apparatus 13 according to the third embodiment in which the exposure control unit 1317 is added to the information processing apparatus 13 of the first embodiment.

Moreover, the effects in each of the embodiments and modifications described in the present specification are merely illustrative and are not restrictive, and other effects are achievable.

### REFERENCE SIGNS LIST

1 MEDICAL SYSTEM
11 LIGHT SOURCE
12 IMAGE CAPTURING APPARATUS
13 INFORMATION PROCESSING APPARATUS
14 DISPLAY APPARATUS
131 PROCESSING UNIT
132 STORAGE UNIT
1311 ACQUISITION UNIT
1312 MOTION DETECTION UNIT
1313 FIRST SC CALCULATION UNIT
1314 SECOND SC CALCULATION UNIT
1315 SC IMAGE GENERATION UNIT
1316 DISPLAY CONTROL UNIT
1317 EXPOSURE CONTROL UNIT

1. A medical system comprising:
light irradiation means for irradiating an image capturing target with coherent light;
image capturing means for capturing a speckle image obtained from scattered light caused by the image capturing target irradiated with the coherent light;
acquisition means for acquiring a first speckle image at a first exposure time and a second speckle image at a second exposure time shorter than the first exposure time;
speckle contrast calculation means for calculating a first speckle contrast value for each pixel based on the first speckle image and/or a second speckle contrast value for each pixel based on the second speckle image;
motion detection means for detecting motion of the image capturing target; and
speckle image generation means for generating a speckle contrast image on a basis of the first speckle contrast value and/or the second speckle contrast value depending on a detection result of the motion of the image capturing target by the motion detection means.

2. The medical system according to claim 1, wherein the medical system is a microscopic surgery system or an endoscopic surgery system.

3. An information processing apparatus comprising:
acquisition means for acquiring a first speckle image at a first exposure time and a second speckle image at a second exposure time as a speckle image obtained from scattered light caused by an image capturing target irradiated with coherent light, the second exposure time being shorter than the first exposure time;
motion detection means for detecting motion of the image capturing target;
speckle contrast calculation means for calculating a first speckle contrast value for each pixel based on the first speckle image and/or a second speckle contrast value for each pixel based on the second speckle image; and
speckle image generation means for generating a speckle contrast image on a basis of the first speckle contrast value and/or the second speckle contrast value depending on a detection result of the motion of the image capturing target by the motion detection means.

4. The information processing apparatus according to claim 3, wherein
the acquisition means acquires a mixed image including a pixel of the first speckle image and a pixel of the second speckle image in one frame, and
the speckle contrast calculation means calculates the first speckle contrast value for each pixel on a basis of the pixel of the first speckle image in the mixed image and calculates the second speckle contrast value for each pixel on a basis of the pixel of the second speckle image in the mixed image.

5. The information processing apparatus according to claim 3, wherein the acquisition means alternately acquires the first speckle image and the second speckle image in a time-series order.

6. The information processing apparatus according to claim 3, wherein the acquisition means acquires the first speckle image and the second speckle image respectively from two image capturing means having different exposure time periods.

7. The information processing apparatus according to claim 3, wherein
the acquisition means acquires a high frame rate speckle image, and
the speckle contrast calculation means calculates the first speckle contrast value by using a plurality of frames of the high frame rate speckle image as the first speckle image, and
calculates the second speckle contrast value by using one frame of the high frame rate speckle image as the second speckle image.

8. The information processing apparatus according to claim 3, wherein
the speckle image generation means
generates the speckle contrast image on a basis of the first speckle contrast value upon no detection of the motion of the image capturing target by the motion detection means, and
generates the speckle contrast image on a basis of the second speckle contrast value upon detection of the motion of the image capturing target by the motion detection means.

9. The information processing apparatus according to claim 3, wherein
the speckle image generation means
generates the speckle contrast image by using a speckle contrast value obtained by weighting and summing and synthesizing the first speckle contrast value and the second speckle contrast value on a basis of an amount of movement of the image capturing target detected by the motion detection means.

10. The information processing apparatus according to claim 3, wherein
the motion detection means
detects the motion of the image capturing target on a basis of a value obtained by subtracting the first speckle contrast value from the second speckle contrast value.

11. The information processing apparatus according to claim 3, further comprising: exposure control means for controlling an exposure time of image capturing means for capturing the speckle image on a basis of the motion of the image capturing target detected by the motion detection means.

12. An information processing method comprising:
an acquisition process of acquiring a first speckle image at a first exposure time and a second speckle image at a second exposure time as a speckle image obtained from scattered light caused by an image capturing target irradiated with coherent light, the second exposure time being shorter than the first exposure time;
a motion detection process of detecting motion of the image capturing target;
a speckle contrast calculation process of calculating a first speckle contrast value for each pixel based on the first speckle image and/or a second speckle contrast value for each pixel based on the second speckle image; and
a speckle image generation process of generating a speckle contrast image on a basis of the first speckle contrast value and/or the second speckle contrast value depending on a detection result of the motion of the image capturing target in the motion detection process.
